SEQUENCE LISTING (3 sequences)

SEQ ID NO: 1 - protein, 5 aa, Artificial Sequence
  5 amino acid fragment of natriuretic peptide
RIDRI

SEQ ID NO: 2 - protein, 12 aa, Artificial Sequence
  12 amino acid fragment of natriuretic peptide with carboxy terminal amine
CFGGRIDRIGAC

SEQ ID NO: 3 - protein, 15 aa, Artificial Sequence
  15 amino acid fragment of natriuretic peptide with carboxy terminal amine
RSSCFGGRIDRIGAC

(19) United States
(12) Patent Application Publication
(10) Pub. No.: US 2011/0300071 A1
(43) Pub. Date: Dec. 8, 2011

(54) TARGETING NPR-C IN ANGIOGENESIS AND ATHEROSCLEROSIS WITH A C-TYPE ATRIAL NATRIURETIC FACTOR (CANF)-COMB NANOCOMPLEX

(75) Inventors: Pamela K. WOODARD, St. Louis, MO (US); Yongjian LIU, Chesterfield, MO (US); Eric PRESSLY, Isla Vista, CA (US); Dana ABENDSCHEIN, St. Louis, MO (US); Craig J. HAWKER, Santa Barbara, CA (US); Michael J. WELCH, Clayton, MO (US)

(73) Assignee: Washington University, St. Louis, MO (US)

(21) Appl. No.: 13/152,933

(22) Filed: Jun. 3, 2011

Related U.S. Application Data
(60) Provisional application No. 61/351,847, filed on Jun. 4, 2010.

(51) Int. Cl. A61K 51/08; A61P 9/10; A61P 7/00

(52) U.S. Cl. 424/1.69

(57) ABSTRACT

Tracers are disclosed comprising an amphiphilic comb-like nanostructure conjugated with an oligopeptide such as a fragment of a natriuretic peptide, and a signaling moiety such as a positron-emitting radionuclide. A fragment of a natriuretic peptide comprises Arg-Ile-Asp-Arg-Ile (SEQ ID NO:1). Further disclosed are methods of imaging distribution of C-type atrial natriuretic peptide receptors and methods of imaging angiogenesis and atherosclerosis by PET scanning or MRI using a tracer.

### CROSS REFERENCE TO RELATED APPLICATIONS

This application claims priority from U.S. Provisional Application Ser. No. 61/351,847, filed Jun. 4, 2010, which is incorporated herein by reference in its entirety.

### GOVERNMENTAL INTEREST

The Invention was made with government support under U.S.P.H.S. Grants U01 HL080729 and HHSN268201000046C awarded by the National Institutes of Health and National Cancer Institute Grant CA86307. The government has certain rights in the invention.

### INCORPORATION BY REFERENCE OF SEQUENCE LISTING

The Sequence Listing, which is a part of the present disclosure, includes a computer readable form and a written sequence listing comprising nucleotide and/or amino acid sequences. The sequence listing information recorded in computer readable form is identical to the written sequence listing. The subject matter of the Sequence Listing is incorporated herein by reference in its entirety.

### INTRODUCTION

Antiangiogenic therapy in conjunction with traditional chemotherapy and radiation represents a major step towards more selective and better-tolerated cancer treatments. However, there remains a need for imaging probes that permit sensitive detection and characterization of tumor angiogenesis and provide a means of following the progress of antiangiogenic tumor treatments (Dijkgraaf I, et al. Cancer Biother Radiopharm. 24:637-647, 2009).

Similarly, there is also a need for imaging probes that permit sensitive detection and characterization of atherosclerosis including atherosclerotic plaque, and provide a means of following the progress of treatments. Currently, most diagnostic modalities used for imaging atherosclerotic plaques assess the severity of the stenosis and/or plaque morphology. These tests include x-ray angiography, computed tomographic (CT) angiography, magnetic resonance imaging and intravascular ultrasound (Nissen, S. E., et al., Circulation 103: 604-616 2001; Saam T, et al., Radiology 244: 64-77 2007; Topol, et al., Circulation 92: 2333-2342 1995; Hong, C., et al., Radiology. 223: 474-480 2002; 2002; Sirol. M, Circulation 109: 2890-2896 2004). However, none of these modalities is able to provide information on the biology and metabolism of the plaque that may predict the rupture. (Fayad, Z. A., et al., Circ. Res. 89:305-316 2001; Fayad, Z. A., Neuroimaging Clin. N. Am. 12: 461-471, 2002.) Several radionuclide-based approaches for non-invasive, functional imaging of atherosclerosis have been developed and evaluated in animal models (Rosen, J. M., et al., J. Nucl. Med. 1990; 31:343-350; Vallabhajosula, S., et al., J. Nucl. Med. 29: 1237-1245, 1988; Rudd, J. H., et al., J. Nucl. Med. 49: 871-878, 2008; Langer. H. F., et al., J. Am. Coll. Cardiol. 52: 1-12, 2008; Vallabhajosula, S., et al., J. Nucl. Med. 38: 1788-1796, 1997; Tan, K. T., et al., Int. J. Cardiol. 127: 157-165, 2008; Ogawa, M., et al., J. Nucl. Med. 45: 1245-1250, 2004; Chang, M. Y., et al., Arterioscler Thromb. 12: 1088-1098, 1992; Kolodgie, F. D., et al., Circulation 108: 3134-3139, 2003; Lees, A. M., et al., Arteriosclerosis. 8: 461-470, 1988; Matter, C. M., et al., Circ. Res. 95: 1225-1233, 2004; Nahrendorf, M., et al., Circulation 117: 379-387, 2008; Prat, L., et al., Eur. J. Nucl. Med. 20: 1141-1145, 1993). Among the tracers for plaque imaging, those containing γ-emitters (technetium 99m, indium 111, iodine 123, etc.) suffer from the limited spatial resolution of single photon emission tomography (SPECT) (Davies, J. R., et al., J. Nucl. Med. 45: 1898-1907, 2004). In contrast, because of superior spatial resolution, positron emission tomography (PET) is more suitable for plaque imaging. (Langer. H. F., et al., J. Am. Coll. Cardiol. 52: 1-12, 2008; Davies, J. R., et al., J. Nuel. Med. 45: 1898-1907, 2004). To date, many PET radiotracers have been evaluated for imaging of atherosclerosis (Davies, J. R., et al., J. Nucl. Med. 45: 1898-1907, 2004). Among them, fluorine-18-fluorodeoxyglucose (FDG) is the most investigated (Rudd, J. H., et al., J. Nucl. Med. 49: 871-878, 2008; Ogawa, M., et al., J. Nucl. Med. 45: 1245-1250, 2004; Rudd, J. H., et al., Circulation 105: 2708-2711, 2002). Uptake of FDG in the aortic wall of patients with atherosclerosis has been attributed to infiltration of macrophages, smooth muscle cells, and lymphocytes within active atherosclerotic lesions (Tawakol, A., et al., J. Nucl. Cardiol. 12: 294-301, 2005). However, FDG accumulates in all metabolically active tissues as well as sites of inflammation and, therefore, its use for specific imaging of atherosclerotic plaques, and especially of vulnerable plaques, requires further evaluation (Laurberg, J. M., et al., Atherosclerosis 192: 275-282, 2007). The biology of atherosclerosis provides a number of other potential biomarkers for plaque imaging. For instance, degradation of the extracellular matrix and cell apoptosis are involved in plaque destabilization and can be imaged by using protease derivatives (cathepsin and matrix metalloproteinases) or radiotracers based on annexin-V (Jaffer, F. A., et al., J. Am. Coll. Cardiol. 47: 1328-1338, 2006). Also, the formation of plaque neovessels has been associated with interplaque hemorrage, cholesterol deposition and plaque growth, and therefore could be a marker of plaque vulnerability. Hence, angiogenesis markers such as, integrins, VEGF, and VCAM-1 are currently under evaluation for vulnerable plaque imaging with PET (Beer, A. J., et al., Cancer Metastasis Rev. 27: 631-644, 2008).

Natriuretic peptides (NPs) are a family of cardiac- and vascular-derived hormones that play a relevant role in cardiovascular homeostasis (Woodard G E, et al. Int rev Cell Mol. Biol. 268:59-93, 2008). Among the four family members, atrial natriuretic peptide (ANP) and C-type natriuretic peptide (CNP) have been demonstrated to suppress the signaling of vascular endothelial growth factor (VEGF), a key regulator of angiogenesis (Dijkgraaf I, et al. Cancer Biother. Radiopharm. 24: 637-647, 2009). Furthermore, ANP has been reported to attenuate the angiogenesis process (Kong, X., et al., Cancer Res. 68: 249-256, 2008; Vesely, D. L., J. Investig. Med. 53: 360-365, 2005). The NPs exert their biological effects through their interaction with NP receptors (NPRs) (Maack, T., et al., Science 238: 675-678, 1987). Among the NPRs, the clearance receptor (NPR-C) constitutes approximately 95% of the entire NPR population. In addition, NPR-C is the only NPR that recognizes all the NPs as well as NP fragments containing as few as five conserved amino acids (Arg-Ile-Asp-Arg-Ile) (Maack, T., Arq. Bras. Endocrinol. Metabol. 50: 198-207, 2006).

Molecular imaging, as an evolving technique, has played a major role in noninvasive, assessment of biologic processes in vivo and drug discovery over the past decade (Rosin R, et al. In: Schuster D P, Blackwell T S, eds. Molecular imaging of the lungs. New York: Taylor and Francis 2005:3-39; Dobrucki L W, et al. Nat Rev Cardiol 7:38-47, 2010; Sinusas A J, et al. Circ Cardiovasc Imaging. 1:244-256, 2008; Rudin M. Curr Opin Chem. Boil. 13:360-371, 2009).

In our previous study, we showed the ⁶⁴Cu labeled 1,4,7,10-tetraazacyclododecane-1,4,7,10-tetraacetic acid (ROTA)-C-type atrial natriuretic factor (CANF) conjugate (⁶⁴Cu-DOTA-CANF) to be suitable as a tracer for PET imaging of NPR-C in the rabbit atherosclerosis model (Liu Y, et al. J Nucl Med. 51:85-91, 2010). Nevertheless, there remains a continuing need for imaging probes in disease processes.

### SUMMARY

Accordingly, the inventors herein have succeeded in devising new tracers which can be used for imaging distribution of natriuretic peptide receptors, including receptors which bind C-type atrial natriuretic factor (CANF). In some embodiments, these tracers can be used for imaging and monitoring angiogenesis during the course of anti-angiogenic treatment of cancer. In other embodiments, these tracers can be used for imaging and monitoring the presence and progression of atherosclerosis, including imaging of atherosclerotic plaque. In various embodiments, the tracers described herein can be used as probes for imaging angiogenesis or atherosclerosis using positron emission tomography (PET), scanning or magnetic resonance imaging (MRI) or other suitable imaging techniques.

Hence, in some embodiments, the present teachings disclose tracer molecules. A tracer of these embodiments comprises an amphiphilic comb-like nanostructure conjugated with a natriuretic peptide or fragment thereof and a signaling moiety. In some aspects, the oligopeptide can have the sequence of a C-type atrial natriuretic peptide or a fragment thereof. In various configurations, such oligopeptides can comprise the sequence Arg-Ile-Asp-Arg-Ile (SEQ ID NO:1). Thus, in some embodiments, the oligopeptide can be a fragment that is less than a full-length natriuretic peptide. A tracer comprising such oligopeptide fragments can be, in various configurations, a tracer which does not induce vasodilation or cause a drop in blood pressure in a subject following administration to the subject in an amount effective for imaging by positron emission tomography (PET) scanning. A tracer comprising such oligopeptide fragments can be, in various configurations, a tracer which does not induce vasodilation or cause a drop in blood pressure in a subject following administration to the subject in an amount effective for imaging by magnetic resonance imaging (MRI) scanning.

In other embodiments, the present teachings disclose imaging methods. In various aspects, these include methods of determining distribution of C-type atrial natiuretic peptide receptors in a subject. The methods include administering to a subject a tracer comprising a) an amphiphilic comb-like nanostructure conjugated with an oligopeptide comprising a fragment of a natriuretic peptide and b) a positron-emitting radionuclide. In various embodiments, the fragment can include the sequence Arg-Ile-Asp-Arg-Ile (SEQ ID NO:1). The method further includes subjecting the subject to positron emission tomography scanning. In various embodiments, the subject can be any mammal, including a human, such as a human in which angiogenesis is being monitored during an anti-angiogenic treatment for cancer, or a human in which means of imaging plaque is desired.

In yet another embodiment, the present teachings include methods of imaging angiogenesis in a subject. The methods include administering to a subject a tracer comprising a) an amphiphilic comb-like nanostructure conjugated with an oligopeptide comprising a fragment of a natriuretic peptide and b) a positron-emitting radionuclide. In various embodiments, the fragment can include the sequence Arg-Ile-Asp-Arg-Ile (SEQ ID NO:1). The method further includes subjecting the subject to positron emission tomography scanning. In various embodiments, the subject can be any mammal, including a human, such as a human in which angiogenesis is being monitored during an anti-angiogenic treatment for cancer.

In yet another embodiment, the present teachings include methods of imaging atherosclerotic plaque in a subject. The methods include administering to a subject a tracer comprising a) an amphiphilic comb-like nanostructure conjugated with an oligopeptide comprising a fragment of a natriuretic peptide and b) a positron-emitting radionuclide. In various embodiments, the fragment can include the sequence Arg-Ile-Asp-Arg-Ile (SEQ ID NO:1). The method further includes subjecting the subject to positron emission tomography scanning. In various embodiments, the subject can be any mammal, including a human, such as a human in which atherosclerosis is being monitored, such as during a stroke or heart attack.

In various embodiments of the present teachings, the oligopeptide can include at least 2 cysteine residues, which can comprise, in various configurations, at least one cystine (i.e., including a disulfide bridge). In some other configurations, the cysteines can be in reduced form (i.e., not including a disulfide bridge).

In some configurations, the oligopeptide comprised by the tracer can be no greater than about 20 amino acids in length. In some configurations, an oligopeptide comprised by a tracer can comprise the sequence Cys-Phe-Gly-Gly-Arg-Ile-Asp-Arg-Ile-Gly-Ala-Cys-NH₂ (SEQ ID NO:2), in which the carboxy terminal cysteine is aminated. In some configurations, the cysteines of this sequence can comprise a disulfide linkage (a cystine).

In various configurations, an oligopeptide of the present teachings can be no greater than 25 amino acids, no greater than 24 amino acids, no greater than 23 amino, acids, no greater than 22 amino acids, no greater than 21 amino acids, no greater than 20 amino acids, no greater than 19 amino acids, no greater than 18 amino acids, no greater than 17 amino acids, no greater than 16 amino acids, no greater than 15 amino acids, no greater than 14 amino acids, no greater than 13 amino acids, no greater than 12 amino acids, no greater than 11 amino acids, or no greater than 10 amino acids in length. In some configurations, the cysteines of these oligopeptides can comprise a cysteine comprising a disulfide bridge, or can be in the reduced, free sulthydryl form. In addition, an oligopeptide of a tracer of the present teachings can further comprise a sequence unrelated to natriuretic peptide. Further, the tracer can include one or more non-peptidyl components such as a polymer such a polyethylene glycol.

Accordingly, in various aspects, the present teachings disclose a tracer that includes an amphiphilic comb-like nanostructure conjugated with an oligopeptide. A tracer can also include a signaling moiety. An oligopeptide moiety of these aspects can comprise a fragment of a natriuretic peptide, wherein the fragment comprises the sequence Arg-Ile-Asp-Arg-Ile (SEQ ID NO:1). An oligopeptide moiety can comprise a cysteine, and, in certain aspects, the oligopeptide can comprise the sequence Cys-Phe-Gly-Gly-Arg-Ile-Asp-Arg-Ile-Gly-Ala-Cys-NH₂, (SEQ ID NO:2). In some configurations, the oligopeptide can be no greater than about 25 amino acids in length.

In some configurations, the oligopeptide moiety can be no greater than 20 amino acids in length, no greater than 19 amino acids in length, no greater than 18 amino acids in length, no greater than 17 amino acids in length, no greater than 16 amino acids in length, no greater than 15 amino acids in length, no greater than 14 amino acids in length, no greater than 13 amino acids in length, no greater than 12 amino acids in length, no greater than 11 amino acids in length, or no greater than 10 amino acids in length. In some configurations, the cysteine residues can comprise a cysteine. In some configurations, the oligopeptide moiety can be a fragment of a natriuretic peptide and consist of the sequence H-Arg-Ser-Ser-c[Cys-Phe-Gly-Gly-Arg-Ile-Asp-Arg-Ile-Gly-Ala-Cys]-NH₂ (SEQ ID NO:3).

In some configurations, the tracer can comprise a signaling moiety that is a radionuclide such as a positron emitter. A positron-emitting radionuclide of these configurations can be, without limitation, carbon-11, nitrogen-13, oxygen-14, oxygen-15, fluorine-18, iron-52, copper-62, copper-64, zinc-62 zinc-63, gallium-68, arsenic-74, bromine-76, rubidium-82, yttrium-86, zirconium-89, technetium-94m, indium-110m, iodine-122, iodine-124, iodine-131, or cesium-137. In some configurations, a radionuclide can be selected from carbon-11, nitrogen-13, oxygen-15, fluorine-18, iron-52, copper-64, gallium-68, yttrium-86, bromine-76, zirconium-89, iodine-123 or iodine-124 or any combination thereof. In other configurations; a positron emitter can be selected from carbon-11, nitrogen-13, oxygen-15, fluorine-18 and copper-64 or any combination thereof. In some configurations, a radionuclide of the present teachings can be copper-64.

In various configurations, a radionuclide of the present teachings can be comprised by a carrier moiety, such as a chelating agent. In some configurations, a carrier moiety can be, without limitation, a dodecanetetraacetic acid such as 1,4,7,10-tetraazacyclo-dodecane-1,4,7,10-tetracetic acid (DOTA, 1,4,7,10-tetraazacyclododecane-N,N′,N,N′-tetraacetic acid).

In some configurations, the signaling moiety of a tracer of the present teachings can be a T I relaxation time-reducing agent, such as gadolinium, manganese or iron. In some configurations, the T1 relaxation time-reducing agent can be a gadolinium.

In some configurations, the signaling moiety of a tracer of the present teachings can be a T2 relaxation time-reducing agent. In some configurations, the T2 relaxation time-reducing agent can be a superparamagnetic iron oxide (SPIO) or an ultrasmall superparamagnetic iron oxide (USPIO).

### BRIEF DESCRIPTION OF THE DRAWINGS

FIG. 1 illustrates a schematic representation of CANF-Comb nanoparticle synthesis and assembly.

FIG. 2 illustrates [¹⁵O] H₂O dynamic imaging of blood flow in a murine hindlimb ischemia (HLI)-induced model of angiogenesis.

FIG. 3 illustrates bio-distribution of ⁶⁴Cu-DOTA-CANF, ⁶⁴Cu-DOTA-Comb, and ⁶⁴Cu-DOTA-CANF-Comb in C57BL/6 mice.

FIG. 4 illustrates PET/CT imaging of ⁶⁴Cu-DOTA-CANF in HLI-induced angiogenesis model.

FIG. 5 illustrates PET/CT imaging of ⁶⁴Cu-DOTA-CANF-Comb and ⁶⁴Cu-DOTA-Comb in the HLI induced angiogenesis model obtained 7 days after ischemia.

FIG. 6 illustrates immunofluorescent staining of endothelial cells and capillary smooth muscle cells.

FIG. 7 illustrates the immunofluorescent co-localization of NPR-C with neovessel endothelial cells and vascular smooth muscle cells in previously ischemic thigh muscle collected 7 days after femoral arterial surgery.

FIG. 8 illustrates competitive PET and immunofluorescent receptor blocking.

FIG. 9 illustrates a schematic diagram of experimental design for the rabbit atherosclerotic plaque studies.

FIG. 10 illustrates blood clearance of ⁶⁴Cu-DOTA-C-ANF in rabbit.

FIG. 11 illustrates Light micrographs of femoral arterial cross-sections from hypercholesterolemic rabbits obtained after injury.

FIG. 12 illustrates specific binding of ⁶⁴Cu-DOTA-C-ANF on injured arteries from rabbits.

FIG. 13 illustrates ⁶⁴Cu-DOTA-C-ANF tracer uptake SUV on injured femoral arteries, non-injured control arteries, and surrounding muscle with the progression and remodeling of atherosclerotic plaques at three time points.

FIG. 14 illustrates target-to-background ratios of tracer uptake at the three time points studies at three time points in a rabbit atherosclerosis model.

FIG. 15 illustrates a representative PET scan showing ⁶⁴Cu-DOTA-C-ANF distribution in a rabbit atherosclerosis model.

### DETAILED DESCRIPTION

The present inventors disclose a tracer and methods of using the tracer in molecular imaging. The tracer includes an amphiphilic comb-like nanostructure conjugated with an oligopeptide that is a natriuretic peptide or fragment thereof. In addition, the tracer includes a signaling moiety. The natriuretic peptide can be a CANF peptide so that the tracer comprises an amphiphilic comb-like nanostructure conjugated with a CANF peptide or fragment thereof.

In some embodiments, an amphiphilic comb-like nanostructure conjugated with a natriuretic peptide or fragment thereof can comprise CANF-comb copolymers. Such comb copolymers are based upon four building blocks: (a) polyethylene glycol (PEG) which is hydrophilic and can confer protein-resistance; (b) methyl methacryate which can serve as a hydrophobic backbone; (c) a chelator for a signaling moiety, such as, for example, 1,4,7,10-tetraazacyclododecane-1,4,7,10-teteraacetic acid (DOTA) for chelation of a positron emitter such as ⁶⁴Cu; and (d) a targeting peptide such as CANF.

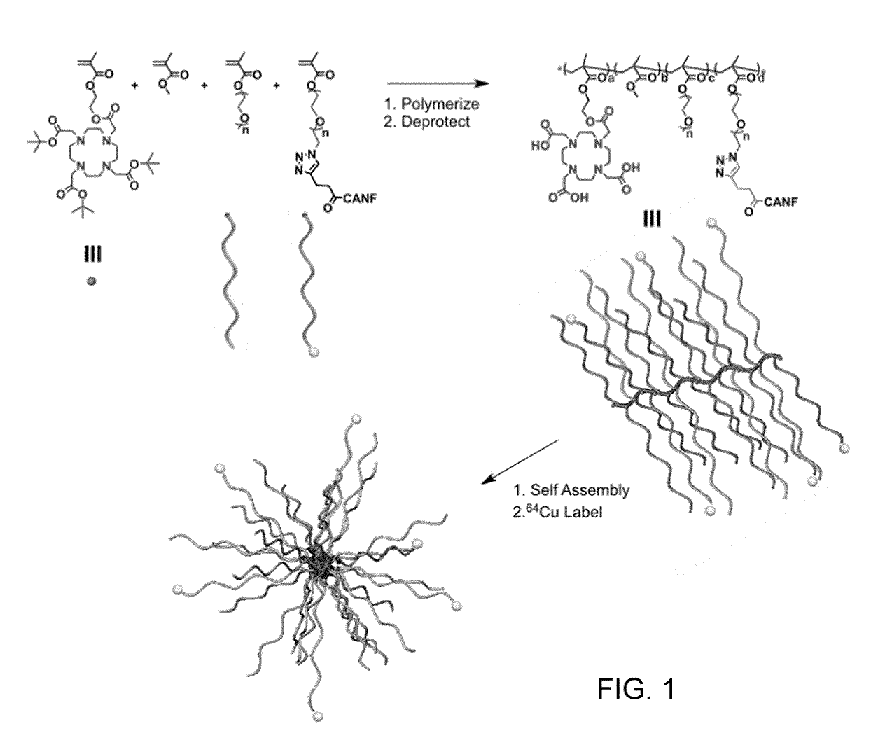

Synthesis of CANF-comb copolymers of the present teachings is described more fully in the Examples below. Briefly the synthesis involved the following. The DOTA methacryate was synthesized from bromomethyllacylate derivative and the tris-functionalized cyclen derivative. This allows direct incorporation of the complex containing the signaling moieties, such as ⁶⁴Cu-DOTA into the interior of the nanoparticle after deprotection and ⁶⁴Cu insertion. The CANF-PEG macromonomer was synthesized in two steps from a heterobifunctional PEG containing a hydroxyl and an azide chain end. The initial step involves introduction of the methacrylate functionality at the hydroxyl end of the heterobifunctional PEG through reaction with methacrylcyl chloride followed by attaching the acetylene-derivitized CANF using Cu(I) click chemistry (Lutz, et al. Angew Chem, Int. Ed. 46:1018-1025, 2007; Parrish, B. et al. J Am Chem Soc 127:7404-7410, 2005; Vestberg R, et al. J Polym Sci, Part A: Polym. Chem. 47:1237-1258, 2009). Using the components described above, the functionalized comb copolymers were synthesized by RAFT polymerization and assembled into comb-like nanoparticles as shown in FIG. 1. Copolymerization of these monomers with varying amounts of PEG methacrylate and methyl methacrylate comonomers allows the preparation of functionalized comb copolymers with varying percent conversion amounting to about 5%, about 10%, about 20%, about 30%, about 40%, up to about 50% or more and, in particular, about 10% as was used in the studies in the Examples below.

The tracers of the present teachings include an oligopeptide moiety which is a natriuretic peptide or fragment thereof (which does not contain the entire amino acid sequence of a full length natriuretic peptide). In various embodiments, the oligopeptide, moiety can comprise the sequence Arg-Ile-Asp-Arg-Ile (SEQ ID NO:1). In various configurations, the oligopeptide moiety can comprise, for example, no more than 25 amino acids of a full length natriuretic peptide, no more than 24 amino acids of a full length natriuretic peptide, no more than 23 amino acids of a full length natriuretic peptide, no more than 22 amino acids of a full length natriuretic peptide, no more than 21 amino acids of a full length natriuretic peptide, no more than 20 amino acids of a full length natriuretic peptide, no more than 19 amino acids of a full length natriuretic peptide, no more than 18 amino acids of a full length natriuretic peptide, no more than 17 amino acids of a full length natriuretic peptide, no more than 16 amino acids of a full length natriuretic peptide, no more than 15 amino acids of a full length natriuretic peptide, no more than 14 amino acids of a full length natriuretic peptide, no more than 13 amino acids of a full length natriuretic peptide, no more than 12 amino acids of a full length natriuretic peptide, no more than 11 amino acids of a full length natriuretic peptide, no more than 10 amino acids of a full length natriuretic peptide, no more than 9 amino acids of a full length natriuretic peptide, no more than 8 amino acids of a full length natriuretic peptide, no more than 7 amino acids of a full length natriuretic peptide, no more than 6 amino acids of a full length natriuretic peptide, or no more than 5 amino acids of a full length natriuretic peptide. The natriuretic peptide can be an atrial natriuretic peptide. In some embodiments, the peptide can be a C-type atrial natriuretic peptide. The natriuretic peptide can be a human atrial natriuretic peptide.

In various embodiments, a signaling moiety of the tracer can be any signaling moiety effective for providing a detectable signal using PET scanning. For PET, a signaling moiety can be any positron-emitting isotope known to skilled artisans. In various embodiments, a signaling moiety of a tracer of the present teachings can be any signaling moiety effective for providing a detectable signal using MRI. In these embodiments, the signaling moiety can be any T1 relaxation time-reducing agent, or any T2 relaxation time-reducing agent known to skilled artisans.

The present inventors disclose PET imaging of NPR-C receptor up-regulation associated with ischemia induced angiogenesis in mice. The NPR-C receptor presence was identified with the ⁶⁴Cu-DOTA-CANF-Comb nanoprobe and PET/CT, as well as the immunohistochemistry. The imaging capability and superiority of the targeted ⁶⁴Cu-DOTA-CANF-Comb nanoprobe over the ⁶⁴Cu-DOTA-CANF peptide tracer were demonstrated.

The ⁶⁴Cu-DOTA-CANF-Comb nanoprobe offers sensitive and targeted molecular imaging for NPR-C expression, for example in a rabbit atherosclerosis model. The superiority of the CANF-comb nanoprobe over the CANF-peptide is demonstrated in the examples below.

PET imaging illustrated significantly (p<0.05) higher standardized uptake values (SUV) of ⁶⁴Cu-DOTA-CANF-Comb nanoprobe at the injured sites relative to the non-injured control site in a rabbit atherosclerosis models. Furthermore, the tracer uptake at the lesion of the targeted nanoprobe was much higher (p<0.05) than that of control nanoprobe. More importantly, in contrast to the previously published ⁶⁴Cu-DOTA-CANF peptide tracer, the ⁶⁴Cu-DOTA-CANF-Comb nanoprobe showed greatly increased (p<0.05) uptake and contrast ratio in targeting NPR-C receptor in atherosclerosis models. Western blot results showed the expression of NPR-C receptor. Both PET and IHC blocking studies confirmed receptor mediated tracer uptake.

A previous report had indicated the high sensitivity and specificity of ⁶⁴Cu-DOTA-CANF for imaging NPR-C receptors in vivo (Liu, Y., et al. J Nucl Med. 51:85-91, 2010). However, its fast pharmacokinetics resulted in limited sensitivity and contrast in this murine angiogenesis model, thus making a CANF-modified nanoparticle a viable candidate for overcoming these difficulties. In related studies, the PMMA-core/PEG-shell amphiphilic nanoparticle showed in vivo behavior that could be accurately tailored by changing the molecular parameters of the starting functionalized copolymer (Welch, M. J., et al. J. Nucl. Med. 50:1743-1746, 2009). Scheme 1 (FIG. 1) shows a schematic representation of the synthetic design starting with a mixture of hydrophilic, hydrophobic, and functional monomers which are copolymerized to form an amphiphilic graft copolymer which on self-assembly leads to tailored nanoparticles. The ability to control both the number and location of functional groups within this nanoscale construct allows a high loading of DOTA macrocycle in the core of the nanoparticle which in turn results in high specific activity for the ⁶⁴Cu-nanoparticle complexes. In our initial exploration of CANF-modified nanoparticles, we chose to use 5 kDa PEG chains to maximize blood circulation lifetime (Pressly, E. D., et al, Biomacromolecules. 8:3126-3134, 2007) with the resulting CANF-PEG macromonomer synthesized through click chemistry (FIG. 1). Copolymerization with non-functionalized PEG macromonomers then gives the desired DOTA-CANF-Comb in which 10% of the PEG chain ends were functionalized with the CANF peptide (˜14 per particle) for initial evaluation. Higher loadings of targeting ligand were found to result in significant lowering of blood retention profiles (Shokeen, M., et al. ACS Nano. 5:738-747, 2011).

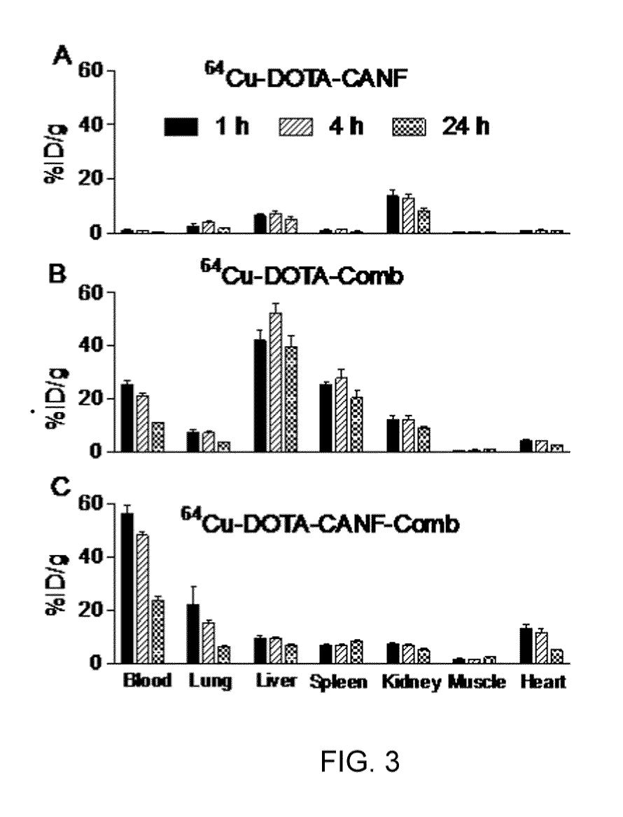

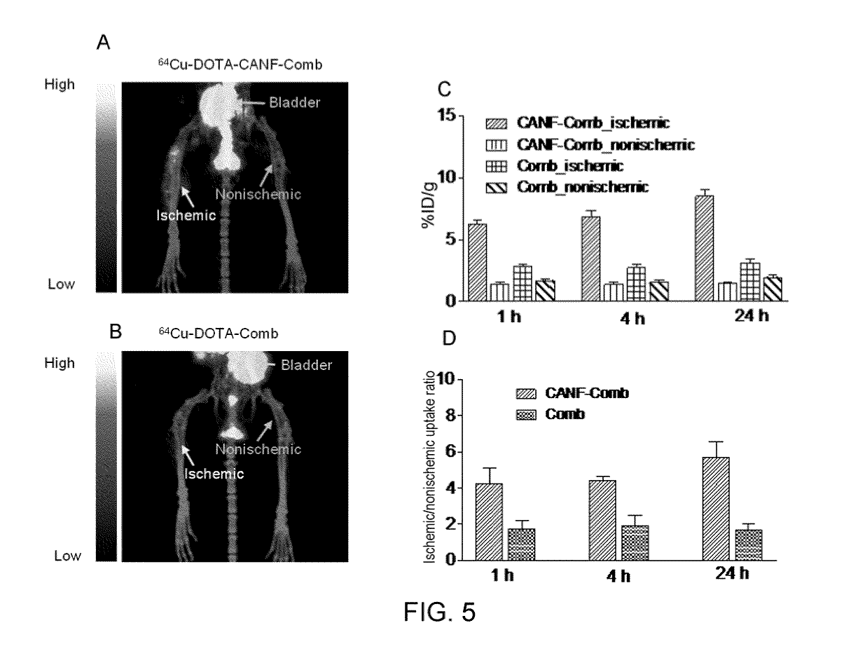

The bio-distribution of the non-targeted control comb showed enhanced blood retention but presumably increased mononuclear phagocytic system (MPS) uptakes (FIG. 3B) compared to the small molecule ⁶⁴Cu-DOTA-CANF peptide tracer alone. (Owens, D. E. 3rd, and Peppas, N. A., Int. J. Pharm. 307:93-102, 2006). Increased accumulation in the ischemic lesion, possibly due to the enhanced permeability and retention (EPR) effect, (Fang, J., et al. Advanced Drug Delivery Reviews 63:136-151, 2011) was observed with the increase being modest and consistent with a non-targeted control nanoprobe. After conjugation with the CANF targeting peptide, the pharmacokinetics of the ⁶⁴Cu-DOTA-CANF-Comb nanoprobe were further improved with decreased MPS clearance and extended retention in blood relative to the non-targeted nanoprobe (FIG. 3B, C), possibly due to the charge effect of nanoparticles (Owens, D. E., et al. Int. J. Pharm. 307:93-102, 2006). Additionally, the high specific activity (5.4±1.2 GBq/nmol) and binding affinity (Maack, T., Arq. Bras. Endocrinol. Metabol. 50:198-207, 2006) of the ⁶⁴Cu-DOTA-CANF-Comb required only 7 picomole of tracer for in vivo administration, leading to high contrast and accurate quantification. Thus, the ⁶⁴Cu-ROTA-CANF-Comb nanoprobe PET imaging showed significantly enhanced tracer uptake in the injured thigh muscle and improved ischemic/nonischemic uptake ratios when compared to the non-targeted nanoprobe during a 24 h study (FIG. 5A, C, D). More importantly, compared to the DOTA-CANF peptide tracer alone, the uptake of ⁶⁴Cu-DOTA-CANF-Comb was 2.2 times higher at 1 h p.i. and increased over time (3.4 at 24 h p.i.) due to the improved blood retention and targeting efficiency, and possibly combined EPR effect. This demonstrates the importance of multivalency in developing sensitive and specific nanoprobe for molecular PET imaging to improve the targeting efficiency and radiolabeling specific activity.

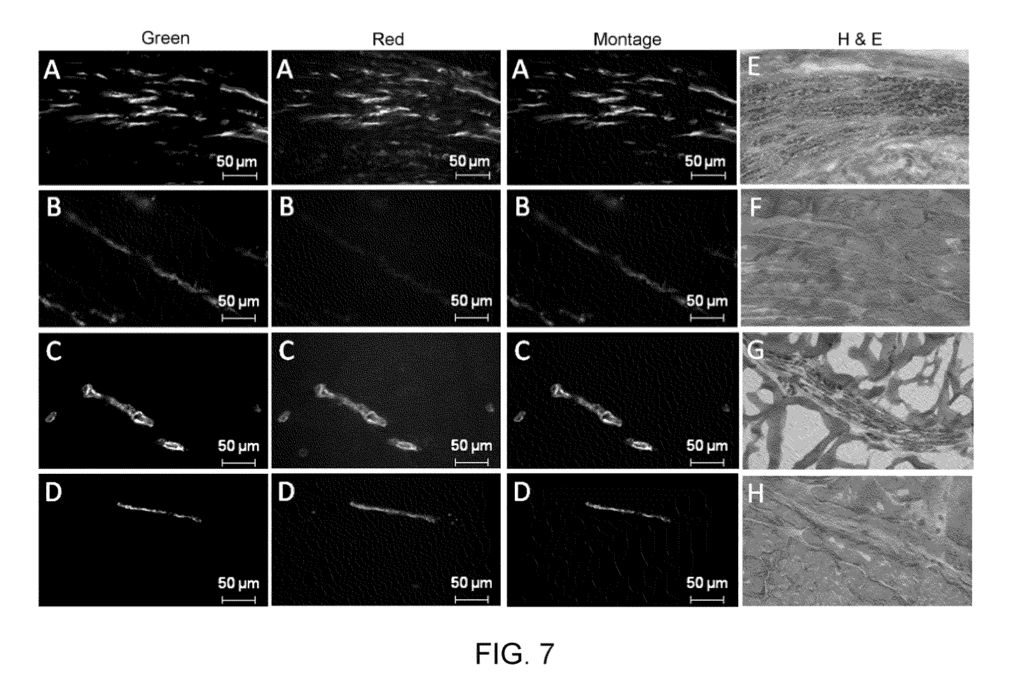

The HLI model has been used for many studies to identify various biomarkers (Limbourg, A., et al. Nat. Protoc. 4:1737-1746, 2009). The [¹⁵O] H₂O PET imaging demoristrated the creation of ischemia and restoration of blood flow 7 days after. The PECAM staining of the previously ischemic thigh muscle showed a tightly packed bundle of newly formed capillaries, confirmed by H&E staining (FIG. 7A, E), relative to the nonischemic thigh tissue (FIG. 7B, F), indicating the presence of angiogenesis. The NPR-C staining showed the increased expression of NPR-C receptor and more importantly the co-localization with PECAM, confirming the up-regulation of NPR-C in the endothelium of neovessels. In addition, the increased expression of NPR-C in smooth muscle cells of previously ischemic compared to nonischemic tissue also corroborated NPR-C as a new bio-marker for angiogenesis. Competitive receptor blocking with, co-administration of excess unlabeled CANF peptide/DOTA-CANF-Comb nanoparticle decreased the uptake of ⁶⁴Cu-DOTA-CANF/⁶⁴Cu-DOTA-CANF-Comb tracers in the ischemic limb to a level similar to that obtained in nonischemic control limb, as well as the ischemic/nonischemic uptake ratios, indicating NPR-C receptor mediated tracer uptake. Furthermore, a similar decrease in receptor signal observed in ex vivo immunohistochemistry staining also confirmed the NPR-C specific uptake.

In summary, through modular construction of a DOTA-CANF-Comb nanoprobe with tailored physical and biological properties, we have demonstrated the usefulness of a multi-valent nanoprobe for targeting NPR-C receptors in the murine hindlimb ischemia model of angiogenesis. The blood retention, high specific activity, elevated targeting efficiency, and favorable uptake demonstrate the advantages of this amphiphilic DOTA-CANF-Comb nanoprobe.

The methods and compositions described herein utilize laboratory techniques well known to skilled artisans, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al., Cells: A Laboratory Manual, Cold Spring Harbor Laboratory Press, Cold Spring Harbor, N.Y., 1998; and Harlow, E., Using Antibodies: A Laboratory Manual, Cold Spring Harbor Laboratory Press, Cold Spring Harbor, N.Y., 1999; and textbooks such as Fledrickson et al., Organic Chemistry 3rd edition, McGraw Hill, N.Y., 1970. Synthesis of tracers, including synthesis of oligopeptides, can be accomplished using routine methods well know to skilled artisans. In some cases, oligopeptides can be obtained from a commercial supplier, such as, for example, (Cys18)-Atrial Natriuretic Factor (4-18) amide (rat; Code H-3134) from Bachem (Torrence, Calif.) Pharmaceutical methods and compositions described herein, including methods for determination of effective amounts for imaging, and terminology used to describe such methods and compositions, are well known to skilled artisans and can be adapted from standard references such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. As used in the present teachings and the appended claims, the singular forms “a”, “an” and “the” are intended to include the plural forms as well, unless the context indicates otherwise:

The following examples are intended to be illustrative of various embodiments of the present teachings and are not intended to be limiting of the scope of any claim.

### EXAMPLES

The examples below illustrate the use of a positron labeled nanoprobe to image the natriuretic peptide clearance receptor in a hind limb ischemia model and demonstrate that nanoparticles can be very effective molecular imaging agents for positron emission tomography. In the studies reported below, a CANF fragment was conjugated to DOTA chelator and comb-like nanoparticle, respectively, to target the NPR-C receptor in murine hindlimb ischemia (HLI) model of angiogenesis.

### Example 1

This example illustrates the preparation of tracer comprising a CANF fragment conjugated to comb-like nanoparticle and DOTA chelator containing Cu as the signaling moiety.

Materials were purchased from Sigma-Aldrich (St. Louis) and used without further purification unless otherwise stated. The ⁶⁴Cu (half-life=12.7 h, β⁺=17%, β⁻=40%) and [¹⁵O] H₂O (half-life=2.07 min, β⁺=99.9%) were produced at the Washington University cyclotron facility according to methods well known in the art (McCarthy D W, et al. Nucl. Med. Biol. 24:35-43, 1997; Herrero P J, et al. Nucl. Med. 47:477-485, 2006). Functionalized polyethylene glycol) (PEG) derivatives were obtained from Intezyne Technologies (Tampa, Fla.). Tris-t-butylester-DOTA, 1,4,7,10-tetraazacyclododecane and DOTA-NHS were purchased from Macrocyclics (Dallas, Tex.). C-ANF (rat ANF(4-23), Des-Gln (Lees, A. M., et al., Arteriosclerosis 8:461-470, 1988)¹, des-Ser (Matter, C. M., et al., Circ. Res. 95:1225-1233, 2004), des-Gly (Nahrendorf, M., et al., Circulation 117:379-387, 2008; Davies, J. R., et al., J. Nucl. Med. 45:1898-1907, 2004); des-Leu (, L., et al., Eur. J. Nucl. Med. 20:1141-1145, 1993) was purchased from Tianma Pharma (Suzhou, China). Centricon tubes were purchased from Millipore (Billerica, Mass.). HiTrap Desalting columns were from GE Healthcare Biosciences (Piscataway, N.J.). Zeba™ desalting spin columns were from Pierce (Rockford, Ill.). Dithiolester RAFT agent, DOTA methacrylate and N-succinimidyl 4-pentynoate were prepared by methods well known in the art (see for example, Liu, Y., J Nucl Med. 51:85-91, 2010; Pressly, ED., Biomacromolecules 8:3126-3134, 2007; Malkoch, M., Macromolecules 38:3663-3678, 2005; Perrier, S., Journal of Polymer Science, Part A: Polymer Chemistry 43:5347-5393, 2005; Shokeen, M., ACS Nano. 5:738-747, 2011).

Polymeric materials were characterized by ¹H and ¹³C nuclear magnetic resonance (NMR) spectroscopy using either a Bruker 200 or 500 MHz spectrometer (Billerica Mass.) with the residual solvent signal as an internal reference. Gel permeation chromatography was performed in dimethylformamide on a Waters system equipped with four 5-μm Waters columns (300×7.7 mm) connected in series with increasing pore size (10², 10³, 10⁴, and 10⁵ Å.) and Waters 410 differential refractometer index and 996 photodiode array detectors (Milford, Mass.). The molecular weights of the polymers were calculated relative to linear PMMA or PEG standards. Infrared spectra were recorded on a Perkin Elmer Spectrum 100 with a Universal ATR sampling accessory (Waltham, Mass.). Fast protein liquid chromatography was performed on GE ÄKTA system (Piscataway, N.J.) equipped with UV and Beckman 170 radio activity detectors (Fullerton) on a Superose 12 10/300 GL size exclusion column (10×300 mm, GE Healthcare Life Sciences, Piscataway, N.J.). An isocratic elution was performed at 0.8 mL/min by using 20 mM 4-(2-hydroxyethyl)-1-piperazineethanesulfonic acid and 150 mM NaCl mixture with neutral pH.

### Synthesis of DOTA-CANF

CANF and DOTA-NHS conjugation and purification were performed following standard procedures well known in the art (Liu, Y., J. Nucl. Med. 51:85-91, 2009; Rossini, R., J. Nucl. Med. 49:103-111, 2008; Sun, X., Biomacromolecules 6:2541-2554, 2005). Briefly, CANF and DOTA-NHS were mixed in 0.1 mmol/L Na₂HPO₄ (pH 7.5) at 4° C. overnight. The DOTA-conjugated CANF was purified by solid-phase extraction (C-18 Sep-Pak cartridges; Waters) and reversed-phase high-performance liquid chromatography (RP-HPLC), respectively. RP-HPLC was performed on a system equipped with a UV/VIS detector (Dionex) and a radioisotope detector (B-FC-3200; BioScan Inc., Washington, D.C.) on a C-18 analytic column (5 mm, 4.6-220 mm; Perkin Elmer). The linear gradient was from 100% H₂O to 65% acetonitrile in 45 min at a flow rate of 1 mL/min and an ultraviolet absorbance at 210 nm. The conjugation efficiency was more than 95%, as determined by RP-HPLC. The presence of 1 DOTA per peptide was confirmed by liquid chromatography-electrospray ionization mass spectrometry on a 2695 separation and Micromass ZQ module (Waters).

### Synthesis of Acetylene-CANF

CANF (59.3 mg, 0.037 mmol) was dissolved in 2 mL anhydrous DMF. 4-pentynoic anhydride (19.2 mg, 0.098 mmol) dissolved in 1.5 mL anhydrous DMF was added dropwise to the solution which was stirred for 2 days. Cold diethyl ether (15 mL) was added to the solution to triturate the product, which was subsequently dissolved in 2 mL of MilliQ water and freeze dried (yield 47.0 mg, 75%); M_{w}(ESI) 1674.73 {M+H⁺] (calc. 1674.80).

Synthesis of poly(ethylene glycol) CANF methacrylate (CANF-PEGMA)

N₃-PEGMA (75.4 mg, 0.015 mmol) and Acetylene-CANF (42.8 mg, 0.025 mmol) Were dissolved in a solution of 1.0 g DMSO and 0.65 g MilliQ water followed by the additions of 50 μL 5 wt % aqueous CuSulfate (0.018 mmol) and 75 μL 5 wt % aqueous NaAscorbate (0.016 mmol), respectively. The mixture was allowed to stir for two days with repeat additions of CuSO₄ (50 μL) and NaAscorbate (75 μL) solutions after one day. The product was purified by washing (10×) with MilliQ water in 15 mL centricon tubes (YM-5) and freeze-dried (yield 48 mg, 41%) (FT-IR, ν (cm⁻¹): 3315, 2881, 1655, 1466, 1342, 1099, 962, 841. GPC Mₙ 6500, PDI 1.1 (PMMA standards, DMF).

### Synthesis of DOTA-CANF-Comb

The DOTA-CANF-Comb and non-targeted DOTA-Comb were synthesized as reported (Shokeen, M., et al., ACS Nano. 2011; 5:738-747, 2011) replacing the RGD-PEGMA with CANF-PEGMA. Mₙ 205 kDa, PDI 1.20 and Mₙ 220 kDa, PDI 1.25 for DOTA-CANF-Comb and control DOTA-Comb respectively, (GPC-DMF, PMMA standards).

### Assembly of Nanoparticles

The t-butyl protecting groups were removed by methods well known in the art (see for example Shokeen M, ACS Nano. 5:738-747, 2011; Pressly E D, Biomacromolecules. 8:3126-3134, 2007. Malkoch M, Macromolecules. 38:3663-3678, 2005). Typically the t-butyl groups of the DOTA functional groups of the copolymers were deprotected by dissolving in a 9:1 v/v mixture of dichloromethane/trifluoracetic acid (DCM/TFA) followed by solvent removal, redissolving in DCM/TFA and precipitation in hexane.

The deprotected polymers were then dissolved in DMSO (1 wt %), a rapid addition of an equal aliquot of water achieved assembly, and DMSO was removed by centrifugal filtration, resulting in particles of 22.0 nm and 20.4 nm (dynamic light scattering) for the targeting DOTA-CANF-Comb (zeta potential: −1.1±2 mV) and non-targeting DOTA-Comb (zeta potential: −35±2 mV) particles, respectively (see FIG. S1).

### Copper-64 Labeling of DOTA-CANF, DOTA-CANF-Comb and DOTA-Comb

Copper-64 (t_{1/2}=12.7 h, β⁺=17%, β⁻=40%) was produced on the Washington University Medical School CS-15 cyclotron by the ⁶⁴Ni (p,n) ⁶⁴Cu nuclear reaction at a specific activity of 1.85-7.40 GBq/γg at the end of bombardment. ¹⁹DOTA-CANF-Comb and control DOTA-Comb (5 μg, about 6 μmol) were labeled with 185 MBq ⁶⁴Cu in 200 μL 0.1 M pH 5.5 ammonium acetate buffer at 80° C. for 1 h with a yield of 60.5±7.3% (n=15). The ⁶⁴Cu-DOTA-CANF-Comb and ⁶⁴Cu-DOTA-Comb were purified by 2 mL zeba spin desalting column after ethylene diamine tetraacetic acid (10 mM in 50 mM pH. 7.4 phosphate buffer) challenge. The radiochemical purity of the labeled nanoprobe was measured by radioactive thin layer chromatography (Washington D.C.).

### Example 2

This example illustrates the preparation of the Murine Hindlimb Ischemia (MHI) Model.

All animal studies were performed in compliance with guidelines set forth by the NIH Office of Laboratory Animal Welfare and approved by the Washington University Animal Studies Committee. Angiogenesis was induced in male C57BL/6 mice by placing two ligatures on a femoral artery above the saphenous branch and separated by 0.5 cm, followed by excision of the intervening segments. The contralateral femoral artery was exposed, but not ligated or excised as a sham control. The double ligation and vascular resection of a femoral arterial segment produced a severe ischemia of the affected hindlimb (HLI) verified by Doppler blood flow measurements (Perimed) in the distal thigh muscle and a significant increase in muscle blood flow (quantified as the ratio of ischemic to nonischemic hindlimb flow) after 7 days, consistent with lack the restoration of flow to the pre-surgery level and flow enhancement induced by angiogenesis (Almutairi, A., et al. Proc. Natl. Acad. Sci. USA. 106:685-690, 2009). Only animals showing this pattern of profound decrease in distal muscle blood flow at day 0 followed by a marked increase in muscle blood flow at day 7 were used in this study. Approximately 70% of mice undergoing HLI surgery showed the required pattern.

### Example 3

This example illustrates [¹⁵O] water PET estimation of blood flow change. PET using [¹⁵O] water offers direct physiological measurement of circulatory parameters for regional blood and vascular volume. In order to measure the blood flow changes caused by the surgical HLI and the resulting angiogenesis, blood flow was determined using [¹⁵O] H₂O. In these experiments, the [¹⁵O] H₂O (half-life=2.07 min, β⁺=99.9%) was produced at the Washington University cyclotron facility according to methods well known in the art (McCarthy, D. W., et al. Nucl. Med. Biol. 24:35-43, 1997; Herrero, P. J., et al. Nucl. Med. 47:477-485, 2006). About 22-37 MBq of [¹⁵O] water was intravenously (i.v.) injected into the same mice (n=4) after HLI surgery (day 0) and again 7 days later (day 7) (24). A 0-5 min dynamic scan was immediately obtained after the i.v. injection of [¹⁵O] H₂O on an Inveon PET/CT system (Siemens Medical Solutions, Malvern, Pa.). The relative blood flow change was evaluated by standard uptake value (SUV) (Liu Y, et al Mol Pharm. 6:1891-1902, 2009).

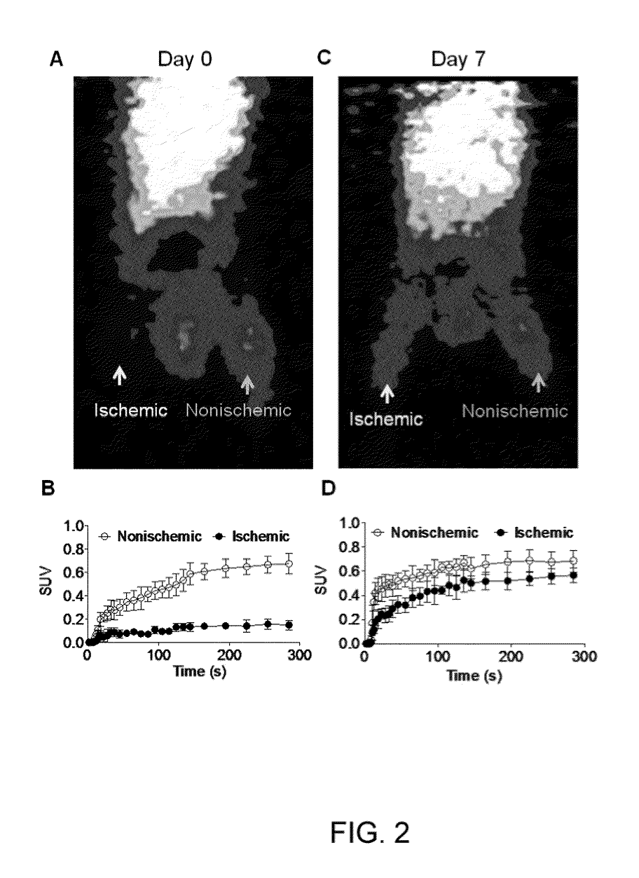

FIG. 2 illustrates the [¹⁵O] H₂O dynamic imaging of blood flow in a murine hindlimb ischemia (HLI)-induced angiogenesis model. FIG. 2A presents a coronal slice on day 0 showing the low blood flow indication of ischemia in the right thigh of a mouse. FIG. 2B presents quantitative standard uptake value (SUV, n=4) of ischemic and nonischemic limbs on day 0; FIG. 2C displays a coronal slice on day 7 showing the recovery of blood flow in the right thigh of mouse shown in FIG. 2A. FIG. 2D presents SUV (n=4) of ischemic and nonischemic lesions on day 7.

After the induction of hind limb ischemia (HLI), blood flow was immediately decreased (FIG. 2A), consistent with the previous report (Limbourg A, et al. Nat. Protoc. 4:1737-1746, 2009). Quantitative dynamic SUVs of the nonischemic and ischemic limbs reached stability at the end of the scan and averaged 0.68±0.09 and 0.15±0.04 (FIG. 2B, n=4, both), respectively; yielding an ischemic/nonischemic SUV ratio of 0.22±0.06 (n=4). On day 7 after HLI surgery, the repeated [¹⁵O] water PET image showed the return of blood flow (FIG. 2C) and the ischemic/nonischemic SUV ratio was increased to 0.83±0.06 (FIG. S2D, n=4, p<0.05), consistent with an angiogenesis-induced increase of blood flow in the previously ischemic tissue.

### Statistical Analysis

The following statistical analyses were used in some experiments reported herein. Group variation is described as mean±standard deviation. Group comparisons were made using 1-way ANOVA with a Bonferroni post-test. Individual group differences were determined with use of a 2-tailed Mann-Whitney test. The significance level in all tests was p<0.05. GraphPad Prism v. 5.02 was used for all statistical analyses.

### Example 4

This example illustrate bio-distribution studies using probes of the present teachings.

In these experiments, ⁶⁴Cu-DOTA-CANF, ⁶⁴Cu-DOTA-CANF-Comb, and Cu-DOTA-Comb were reconstituted in 0.9% sodium chloride (APP pharmaceuticals) for i.v. injection. Male C57BL/6 mice weighing 20-25 g (n=4) were anesthetized with inhaled isoflurane and about 370 kBq of labeled nanoparticles (0.8-1.2 μg/kg body weight) or DOTA-CANF peptide (0.8-1.1 μg/kg body weight) in 100 μL saline were injected via the tail vein. The mice were re-anesthetized before euthanizing them by cervical dislocation at each time point (1 h, 4 h, and 24 h) post injection (p.i.). Organs of interest were collected, weighed, and counted in a well gamma counter (Beckman 8000). Standards were prepared and measured along with the samples to calculate the percentage of the injected dose per gram of tissue (% ID/gram) (Liu, Y., et al. Mol Pharm. 6:1891-1902, 2009).

Biodistribution data of ⁶⁴Cu-DOTA-CANF; Cu-DOTA-Comb, and ⁶⁴Cu-DOTA-CANF-Comb are presented in FIG. 3. The bio-distribution of ⁶⁴Cu-DOTA-CANF, ⁶⁴Cu-DOTA-Comb, and ⁶⁴Cu-DOTA-CANF-Comb in C57BL/6 mice (n=3-4/group) show that: ⁶⁴Cu-DOTA-CANF exhibits fast renal clearance and low blood retention (FIG. 3A); non-targeted ⁶⁴Cu-DOTA-Comb nanoparticle exhibits improved blood retention but high liver and spleen uptakes (FIG. 3B); and the ⁶⁴Cu-DOTA-CANF-Comb nanoprobe exhibits superior pharmacokinetics relative to both CANF peptide tracer alone and the non-targeted control nanoprobe (FIG. 3C).

These data show a fast clearance profile of ⁶⁴Cu-DOTA-CANF primarily through the kidney with minor accumulation in liver, lung and negligible uptake in other organs (FIG. 3A). At 1 h p.i., the blood retention of ⁶⁴Cu-DOTA-CANF was only 0.92±0.45% ID/g, while kidney was 13.7±2.06% ID/g (FIG. 3A). The liver uptakes were constant over 24 h with less than 8% ID/g.

In contrast, the non-targeted ⁶⁴Cu-DOTA-Comb displayed increased blood retention and slower clearance (25.4±3.04% ID/g at 1 h p.i., p<0.001, n=4) (FIG. 3B) compared to ⁶⁴Cu-DOTA-CANF. However, liver uptake was dominant during the 24 h study with more than 40% ID/g at 1 h p.i., and peaked at 4 h p.i. for 52.4±6.85% ID/g. The spleen accumulation showed a similar pattern to the liver with maximum of 27.8±6.52% ID/g at 4 h p.i. However, the kidney uptake was similar to ⁶⁴Cu-DOTA-CANF tracer during the study.

The targeted ⁶⁴Cu-DOTA-CANF-Comb showed a superior bio-distribution profile with significantly improved circulatory retention (blood, lung, and heart) and reduced liver and renal clearance (FIG. 3C). Among the organs, the highest uptake was observed in blood with 56.4±7.54% ID/g, 48.2±2.31% ID/g, and 23.8±2.40% ID/g at 1 h, 4 h, and 24 h p.i. (n=4 for all). All were significantly higher (p<0.001, n=4) than for either the control DOTA-Comb or the CANF peptide tracer alone. Furthermore, liver and spleen accumulations were significantly reduced (p<0.001, n=4) to less than 10% ID/g during the 24 h study when compared to the control DOTA-Comb nanoprobe.

### Example 5

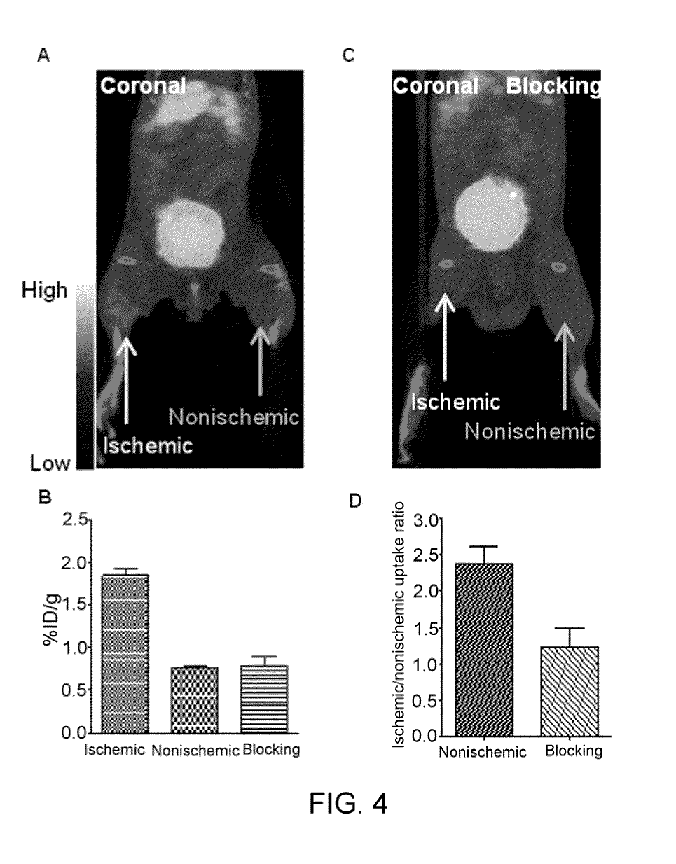

This example illustrates PET/CT imaging of ⁶⁴Cu-DOTA-CANF in the HLI-induced angiogenesis model (FIG. 4).

FIG. 4A presents a coronal slice showing accumulation of ⁶⁴Cu-DOTA-CANF tracer at the injured limb on day 7. FIG. 4B illustrates uptake of ⁶⁴Cu-DOTA-CANF at the ischemic (n=6) and nonischemic (n=6) limbs, as well as the blocking study (n=4). FIG. 4C illustrates competitive receptor blocking by co-administration of unlabeled. CANF peptide, which significantly blocked the tracer uptake. FIG. 4D presents ischemic/nonischemic uptake ratios of non-blocking and blocking studies (n=4, both).

In these experiments, mice showing an increase in blood flow above, baseline level at 7 days after HLI surgery (n=6, 8, and 7 for DOTA-CANF, targeted DOTA-CANF-Comb nanoprobe, and non-targeted Comb, respectively) were anesthetized with isoflurane and injected i.v. with 3.7 MBq/100 μL of activity via the tail vein (8-11 μg/kg and 8-12 μg/kg of mouse body weight for the peptide and nanoprobes, respectively. The Molar ratio of ⁶⁴Cu-DOTA-CANF-Comb to ⁶⁴Cu-DOTA-CANF injected was 100:1.). For ⁶⁴Cu-DOTA-CANF, a 0-60 min dynamic scan was performed on microPET Focus 120/220 (Siemens Medical Solutions) and the microCAT II (CTI-Imtek) scanners. The microPET images (corrected for attenuation, scatter, normalization and camera dead time) and microCT images were co-registered with fiducial markers attached to the animal bed and analyzed using AMIRA (Mercury Computer Systems, Chelmsford, Mass.). For ⁶⁴Cu-DOTA-CANF-Comb and ⁶⁴Cu-DOTA-Comb nanoprobes, the imaging sessions were carried out on an Inveon PET/CT system (Siemens Medical Solutions) and microPET Focus 220 at 1 h, 4 h (one 30-min frame, both) and 24 h p.i. (one 60-min frame). All the PET scanners were cross-calibrated periodically. The microPET images were analyzed with ASIPro (Almutairi, A., et al Proc Natl Acad Sci USA. 106:685-690, 2009). The tracer uptake values were not corrected for partial volume effects (Liu, Y., et al. J Nucl Med 51:85-91, 2010).

After the PET imaging, the animals were euthanized by exsanguination and the thigh containing the previously ischemic and nonischemic control muscles were perfusion fixed in situ with freshly prepared Michel's transport medium (American MasterTech Scientific Inc.) for histopathology and immunohistochemistry.

PET/CT imaging with ⁶⁴Cu-DOTA-CANF at 7 days after HLI surgery showed tracer uptake in the distal thigh muscle, where ischemia had been induced previously, with weak signal deposited in the control, nonischemic limb (FIG. 4A). The uptake of ⁶⁴Cu-DOTA-CANF in the previously ischemic limb was 1.85±0.19% ID/g (n=6); significantly higher (p<0.001) than that obtained in the nonischemic control limb (0.77±0.03% ID/g, n=6, FIG. 4B). With competitive receptor blocking, the tracer uptake of the ischemic limb was reduced to a level similar to that acquired in the nonischemic limb (FIG. 4B,C); significantly lower (p<0.001, n=4) than the uptake before blocking. The ischemic/nonischemic uptake ratio was also decreased from 2.34±0.40 (n=6) to 1.24±0.26 (n=4, p<0.001) after blocking (FIG. 4D).

### Example 6

This example illustrates PET/CT imaging of ⁶⁴Cu-DOTA-CANF-Comb and ⁶⁴Cu-DOTA-Comb in the HLI induced angiogenesis model obtained 7 days after ischemia, as shown in FIG. 5.

FIG. 5A shows distribution of ⁶⁴Cu-DOTA-CANF-Comb in HLI model. Activity accumulated in the ischemic limb with little observed on the contralateral nonischemic limb. FIG. 5B shows distribution of ⁶⁴Cu-DOTA-Comb in HLI model, showing weak uptake in both ischemic and nonischemic limbs. FIG. 5C illustrates uptake of ⁶⁴Cu-DOTA-CANF-Comb (n=8) and ⁶⁴Cu-DOTA-Comb (n=7); and FIG. 5D illustrates ischemic/nonischemic uptake ratios of ⁶⁴Cu-DOTA-CANF-Comb (n=8) and ⁶⁴Cu-DOTA-Comb (n=7).

With targeted ⁶⁴Cu-DOTA-CANF-Comb nanoprobe, an increased accumulation at the lesion site of the ischemic limb was observed (FIG. 5A). The uptake was 6.30±1.07% ID/g (n=8) 1 h p.i., significantly higher (p<0.001) than that obtained (1.40±0.52% ID/g, n=8) in the contralacteral nonischemic limb (FIG. 5A, C), and more importantly, higher (p<0.001) than either the non-targeted ⁶⁴Cu-DOTA-Comb (FIG. 5B, C) or the ⁶⁴Cu-DOTA-CANF tracer (FIG. 4A, B). During the 24 h study, the uptake of ⁶⁴Cu-DOTA-CANF-Comb in the ischemic limb gradually increased to 8.50±1.38% ID/g in contrast to the constant SUV obtained in the contralateral nonischemic limb (FIG. 5C). Moreover, the targeted ⁶⁴Cu-DOTA-CANF-Comb showed significantly higher (p<0.001, n=8) ratio of 4.35±0.87 at 1 h p.i. and 5.35±0.68 at 24 h p.i. than those obtained in comparison to either control ⁶⁴Cu-DOTA-Comb probe (FIG. 5D) or ⁶⁴Cu-DOTA-CANF (FIG. 4D).

### Example 7

This example illustrates histopathology and immunohistochemistry.

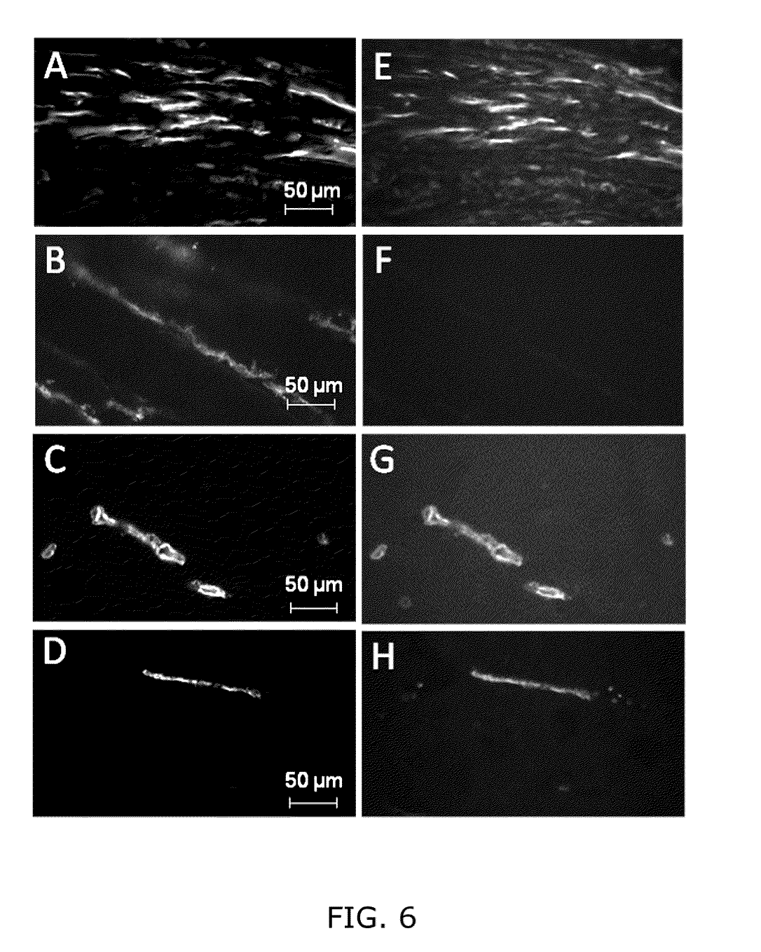

As shown in FIG. 6, illustrates immunofluorescent staining for PECAM-1 in endothelial cells (A, B) or α-actin in capillary smooth muscle cells (C, D) (green in original color images) showing immunofluorescent staining for NPR-C (red in original color images) in endothelial cells (E, F) and smooth muscle cells (G, H).

In these experiments, the perfusion-fixed tissue was stored overnight at 4° C. in Michel's Transport Medium before being frozen in OCT and step-sectioned (7 μm) at 100 μm intervals on a cryostat. Some of the sections at each step were stained with hematoxylin and eosin for identification of the morphology of the tissue.

Tissue sections at 100 μm intervals were also prepared for doubles immunofluorescent staining of NPR-C and either PECAM-1 for endothelial cells or alpha actin for vascular smooth muscle cells. The method for double immunolabeling followed the manufacturer's recommendations (Vector Laboratories). Briefly, the steps included avidin/biotin blocking; protein blocking with 5% normal horse serum for the first antibody (MEC 13.3 against CD31 [PECAM-1] or monoclonal anti-actin isotype IgG2a) and 5% normal goat serum for the second antibody (ANPC antibody [N-term] purified rabbit monoclonal); incubation for 30 min with the primary antibody diluted 0.04-0.5 mg/mL in buffer with normal serum added; incubation for 30 min with the biotinylated secondary antibody diluted 15 μg/mL in buffer with normal serum added; and incubation for 10 min with Fluorescin Avidin DCS (FITC, 10 μg/mL in buffer) for the first antibody (PECAM-1 or alpha actin) and Texas Red Avidin DCS (Rhodamine, 10 μg/mL in buffer) for the second antibody (NPR-C). Blocking of primary antibody binding to NPR-C were performed by pre-incubation of diluted antibody with the cognate peptide (0.5 mg/mL) overnight at 4° C. before immunohistochemistry staining. Slides were coverslipped and observed under the fluorescence microscope (Carl Zeiss) with appropriate filters.

### Example 8

This example, as shown in FIG. 7, illustrates the immunofluorescent co-localization of NPR-C with neovessel endothelial cells and vascular smooth muscle cells in previously ischemic thigh muscle collected 7 days after femoral arterial surgery showing (FIG. 7A, C, E, G) Fluorescent and light images of previously ischemic hindlimb tissue; (FIG. 7B, D, F, H) Fluorescent and light images of contralateral nonischemic hindlimb tissue. Co-registration (FIG. 7A, C, D) (orange in original color images) of fluorescent images for PECAM-1 (green in original color images) or α-actin (green in original color images) to NPR-C (red in original color images). FIG. 7E: hematoxylin and eosin (H&E) staining showing a band of neovessels cut in longitudinal section corresponding to the location of fluorescent staining for endothelium and NPR-C in panel A. FIG. 7G): H&E staining showing coagulation necrosis of muscle and stained nuclei (blue in original color images) of a neovessel (center) and in previously ischemic tissue corresponding to the location of fluorescent staining for smooth muscle cell and NPR-C (panel C). Interestingly, neovessels in the previously ischemic tissue and existing capillaries in the nonischemic tissue both stained for α-actin and NPR-C although the staining was much fainter in nonischemic tissue FIG. 7D. Scaling line shows 50 μm.

Thigh muscle from the previously ischemic hindlimb showed areas of coagulation necrosis, but also an abundance of new capillaries, which in some sections appeared as tightly packed bundles identified by PECAM-1 staining of endothelial cells and by hematoxylin and eosin-staining (Couffinhal, T., et al. Am Pathol. 152: 1667-1679, 1998). Moreover, double immunostaining for PECAM-1 (FIG. 6A) and NPR-C (FIG. 6C) showed co-localization of NPR-C (red in original color images) and PECAM-1 (green in original color images) on the endothelium of neovessels (FIG. 7A, E). The increased density of neovessels reflecting angiogenesis in previously ischemic hindlimbs was not observed in the tissue from the sham-operated, nonischemic limb. Very little NPR-C staining was observed in the tissue from the sham-operated limb suggesting the presence of NPR-C primarily on the endothelium of neovessels rather than established capillaries (FIG. 7 B, F). Vascular smooth muscle cells identified with an antibody to α-actin (green in original) also exhibited staining for NPR-C (FIG. 7 C, G). Interestingly, both neovessels in the previously ischemic limb and existing capillaries in the nonischemic limb showed co-localization of NPR-C and α-actin which was unlike the selectivity shown for the endothelium of neovessels although there was more fluorescent signal from the ischemic tissue.

### Example 9

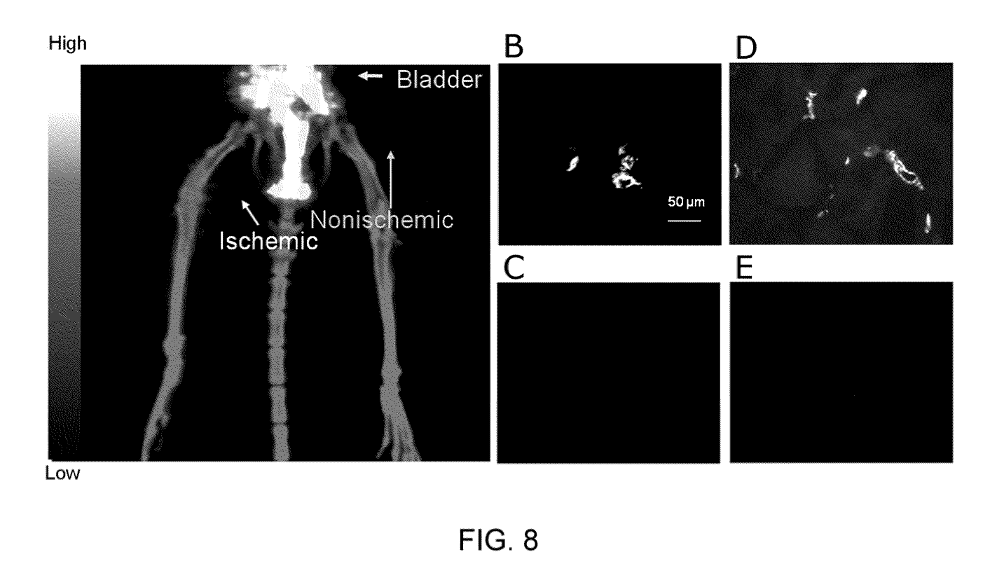

This example (FIG. 8) illustrates competitive PET and immunofluorescent receptor blocking showing: (A) ⁶⁴Cu-DOTA-CANF-comb in HLI mice with co-administration of unlabeled DOTA-CANF-comb showing the significantly reduced accumulation at ischemic limb; (B) fluorescent images of ischemic thigh muscle stained with NPR-C on endothelia; (C) immunofluorescent staining for NPR-C after competitive blocking of antibody-antigen binding showed receptor specific binding; (D) fluorescent images of ischemic thigh muscle stained with NPR-C on smooth muscle cells; and (E) immunofluorescent staining for NPR-C after competitive blocking of antibody-antigen binding showed receptor specific binding. (Scaling line shows 50 μm).

Competitive receptor blocking studies were performed in mice with HLI surgery (27±2.5 g) for ⁶⁴Cu-DOTA-CANF by co-injection of unlabeled CANF peptide (CANF: ⁶⁴Cu-DOTA-CANF=100:1 mole ratio, n=4) on day 7 after the surgery immediately followed by 0-60 min dynamic scans. For the ⁶⁴Cu-DOTA-CANF-Comb, eight HLI mice (28±3.1 g) received co-injection of unlabeled DOTA-CANF-Comb nanoparticle and ⁶⁴Cu-DOTA-CANF-Comb with 500:1 mole ratio on day 7 after the surgery and were scanned with PET/CT at 1 h, 4 h and 24 h p.i.

Competitive receptor blocking with co-injection of unlabeled DOTA-CANF resulted in a significant uptake decrease in the ischemic region (FIG. 4 C, D). Quantitative uptakes of ⁶⁴Cu-DOTA-CANF-Comb uptake in the lesion site 7 days after the injury (3.34±0.23, 3.34±0.18, 3.31±0.30 at 1 h, 4 h and 24 h time points, respectively, n=6 for all) were significantly (p<0.001) reduced to levels similar to the non-targeted ⁶⁴Cu-DOTA-Comb (2.82±0.47, 2.71±0.79, 3.10±0.94, p>0.05 for all three time points, n=6 for all) (FIG. 8A). Additionally, receptor blocking reduced the ⁶⁴Cu-DOTA-CANF-Comb uptake ratios to the level of the non-targeted DOTA-Comb (1.93±0.46 vs. 1.76±0.41, 1.87±0.22 vs. 1.89±0.59, 1.82±0.55 vs. 1.64±0.40 at 1 h, 4 h and 24 h p.i., n=6 for all). Furthermore, competitive immunohistochemical blocking resulted in loss of the fluorescent signal for NPR-C in both endothelial and smooth muscle cells (FIG. 8B, C, D, E).

### Example 10

This example illustrates materials and methods used in rabbit atherosclerotic-like lesion studies.

### Animal Preparations to Induce Atherosclerotic-Like Lesions

All animal studies were performed in compliance with guidelines set forth by the NIH Office of Laboratory Animal Welfare and approved by the Washington University Animal Studies Committee. Complex atherosclerotic-like arterial lesions containing a fibrous cap and a lipid-enriched core, similar to the structure of atheromatous plaques in human arteries, were induced in the right femoral artery of rabbits. Injury was induced by air desiccation and followed by angioplasty at a later time point as reported previously (Sarembock, I. J., et al., Circulation 80:1029-1040, 1989). Briefly, male New Zealand White rabbits were fed 0.25% cholesterol-enriched diet throughout the study and elevated serum cholesterol (>200 mg/dL) was confirmed at the time of vessel injury. The right femoral artery was exposed aseptically through a longitudinal skin incision and lidocaine was applied topically to prevent spasm. A 1-2 cm segment of the vessel was isolated between air-tight ligatures and small branches were ligated with suture. A 27-gauge needle was used to puncture the isolated segment proximally as a vent. A second 27-gauge needle was inserted distally into the segment and nitrogen gas was passed through the vessel at a flow rate of 80 mL/min for 8 min to dry and cause sloughing of the endothelium. The segment was then flushed with saline and the ligatures were released to restore blood flow, with gentle pressure applied to the puncture sites for a few minutes to maintain hemostasis. The skin incision was closed and the animal was recovered from anesthesia.

Four to six weeks after the air dessication-induced injury, the lesion site and extent of stenosis in the femoral artery were identified by an angiogram obtained with use of a 4F guide catheter introduced through a carotid arterial cutdown and advanced to the distal aorta. Heparin (100 U/kg, intravenous) was given to prevent clot formation in the catheters. A 0.014 in guidewire was then advanced across the lesion and the guide catheter was removed. A 2.0-2.5×20 mm coronary angioplasty balloon was advanced over the guidewire and the site of stenosis was dilated with three, 30 s balloon inflations of 6-8 atm with 1 min between inflations. After re-injuring the lesion site, patency of the femoral artery was confirmed by an angiogram through the angioplasty catheter before the catheter was removed. The carotid was ligated, the skin incision closed, and the animal was recovered from anesthesia.

The left femoral artery remained uninjured as a control.

### Imaging Protocol

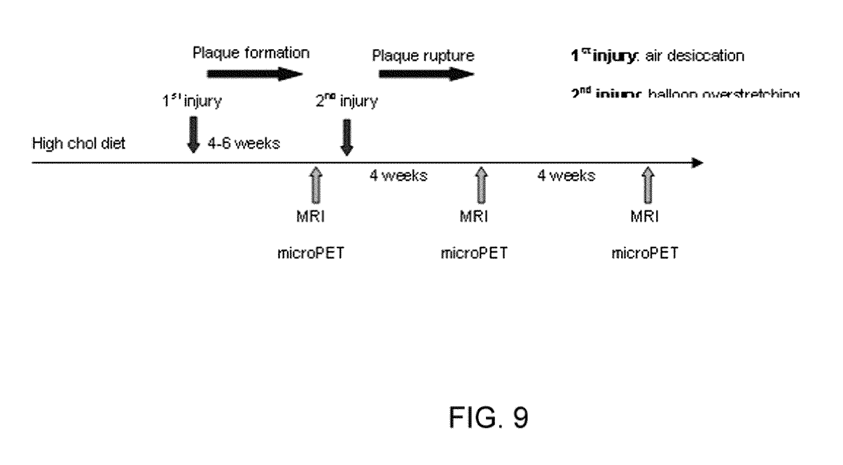

The experimental design is schematized in FIG. 9. Tracer uptake: 8 rabbits (weight=4.1±0.5 kg at the time of the first imaging) were imaged by MRI and small animal PET four-to-six weeks after the air dessication induced-injury (time point (TP) 1); 5 rabbits coming from TP 1 Were imaged by MRI and small animal PET three weeks after the balloon overstretching induced injury (TP 2); 4 rabbits coming from TP 2 were imaged by MRI and small animal PET four weeks after TP 2 (TP 3). For each PET imaging, about 128±32 MBq (n=21) of purified ⁶⁴Cu-DOTA-C-ANF tracer (about 2 nmol) was administered.

### Receptor Blocking Studies

Receptor blocking studies were performed on three additional rabbits (4.1±0.34 kg). One-to-three weeks after the second injury, the animals were imaged with MRI and PET for a pre-blocking study to confirm the presence of atherosclerotic lesions and uptake of ⁶⁴Cu-DOTA-C-ANF to NPR-C receptor on the plaque. One week later, besides the MRI imaging, the PET blocking studies were performed on the same rabbits scanned in pre-blocking studies by co-injection of ⁶⁴Cu-DOTA-C-ANF with a blocking dose of unlabeled C-ANF peptide (1 mg, C-ANF: ⁶⁴Cu-DOTA-C-ANF=100:1 mole ratio) and imaged with PET.

The baseline tracer uptake was measured in one healthy rabbit (4.6 kg) on a normal rabbit chow diet. The level of cholesterol in plasma was analyzed in each rabbit before imaging sessions. Samples of injured and control arteries were taken at each TP for histology and immunohistochemistry.

### MRI Studies

The presence of atherosclerotic lesions in the rabbits was confirmed by 3T MRI 1 hour after administration of a non-receptor specific plaque-targeting contrast agent (Gadofluorine M, 0.5 μmol/kg body weight, Schering AG, Germany) (Sirol, M., et al., Circulation 109:2890-2896, 2004; Zheng, J., et al., Invest. Radiol. 43:49-55, 2008; Meding, J., et al., Mol. Imaging 2:120-129, 2007). This agent has been shown to bind to extracellular matrix proteins such as collagen and proteoglycans (Meding, J., et al., Mol. Imaging 2:120-129, 2007). For scanning, the rabbit was placed supine into a plastic bed. Three micropipette tubes filled with 0.5 mL Gadofluorine M served as fiducial markers, and were taped into position on the bed. At the beginning of the PET study, these tubes were drained of the Gadofluorine by syringe, and refilled with 0.5 mL of ⁶⁴Cu to serve as the PET fiducials. These helped to “co-localize” the plaque regions between the two image modalities.

### PET Studies

Immediately after the MR scan, the rabbits were injected with ⁶⁴Cu-DOTA-C-ANF (3.9±0.9 mCi) and 60 min dynamic scans were acquired on the microPET Focus-220 (Siemens Medical Solutions, Inc., Malvern, Pa.). Fiducial markers attached to the animal bed and filled with a ⁶⁴Cu aqueous solution were used to correlate the MRI and MAP reconstructed PET images. In the competitive blocking experiments, 1 mg of C-ANF was co-administered with the radiotracer (100:1 mole ratio of blocking C-ANF to ⁶⁴Cu-DOTA-C-ANF).

Data analysis of the microPET images was performed using the manufacturer's software (ASI Pro). The accumulation of ⁶⁴Cu-DOTA-C-ANF at the injury site and on the contralateral, non-injured femoral artery (control) was calculated as standardized uptake values (SUVs) in 3D regions of interest (ROIs) by averaging the activity concentration corrected for decay over the contained voxels (multiple image slices) at selected time points post injection. (Sun, X., et al., Bioconjug Chem. 16:294-305, 2005). SUVs were not corrected for partial volume effects.

$$S U V = \frac{Radioactivity concentration in R O I ( μ Ci / {cm}^{3} )}{Injection dose ( μ Ci ) / animal weight ( g )}$$

After the last PET imaging, the animals were euthanized by exsanguination and the femoral vessels were perfusion-fixed in situ with freshly prepared 4% paraformaldehyde. Tissue samples containing the injured and control arteries were harvested for histology and immunohistochemistry.

### Histopathology

Vessel specimens were embedded in paraffin, step sectioned (10 μm) transversely at 1 mm intervals to approximate the distance between MRI slices, and the sections stained with hematoxylin and eosin (H&E) and Verhoeff's Van Gieson (VVG) stain for elastin. The sections were examined to identify the plaque components including foam cells, and vascular smooth muscle cells.

### Immunohistochemistry

Immunohistostaining was performed on paraffin embedded sections from both the injured and non-injured arteries in each rabbit. For immunohistochemistry, we used anti-C-type natriuretic peptide receptor antibody (Abgent, San Diego, Calif.; 1:100) revealed by a secondary fluorescein isothiocyanate-conjugated anti-rabbit antibody (Invitrogen, Carlsbad, Calif.; 1:1000). Slides were viewed with a laser scanning microscope (LSM510 META, Carl Zeiss, Jena, Germany) and the image browser (Carl Zeiss, Jena, Germany). Blocking studies for NPR-C were performed by competitively blocking the primary antibody binding by pre-incubation of diluted antibody (NPR-C rabbit, Abgent, San Diego) with the cognate peptide (0.5 mg/mL) overnight at 4° C. prior to IHC staining. Also, absence of primary antibody was used as a negative control.

### Statistical Analysis

Results are expressed as mean and standard deviation (SD). The 2-tailed paired and unpaired Student's t test were used to test differences within animals (injured artery vs. control artery) and between animals imaged at different time points (such as TP 1 vs. TP 2), respectively. The significance level in all tests was ≦0.05. GraphPad Prism 4.0 was used for all statistical analyses.

### Example 11

This example illustrates copper-64 labeling and serum stability of ⁶⁴Cu-DOTA-C-ANF. With C-18 Sep-Pak purification, the radiochemical purity of the ⁶⁴Cu-DOTA-C-ANF was higher than 98% confirmed by radio-HPLC. The mass spectrometry of the decayed ⁶⁴Cu-DOTA-C-ANF showed one DOTA conjugated to one C-ANF peptide.

⁶⁴Cu-DOTA-C-ANF was highly stable in rabbit serum. The radio-HPLC analysis showed 97.7±3.9% (n=3) intact tracer after 1 h incubation at 37° C. On the contrary, only free ⁶⁴Cu was detected in the control samples (⁶⁴Cu-acetate incubated in rabbit serum) by radio-HPLC.

### Example 12

This example illustrates a tracer blood clearance study using the rabbit model for atherosclerosis.

In these experiments, the blood clearance studies were performed in normal rabbits (n=4) to evaluate the pharmacokinetics of tracer in vivo. About 20 MBq of ⁶⁴Cu-DOTA-C-ANF was injected intravenously into the left ear of rabbit, and blood sample (0.2 mL) was drawn from the contralacteral ear over the period of 1 h (1 min, 3 min, 5 min, 10 min, 20 min, 40 min and 60 min). The activity of the blood samples were counted in gamma counter and presented in percent injected dose per gram (ID %/g).

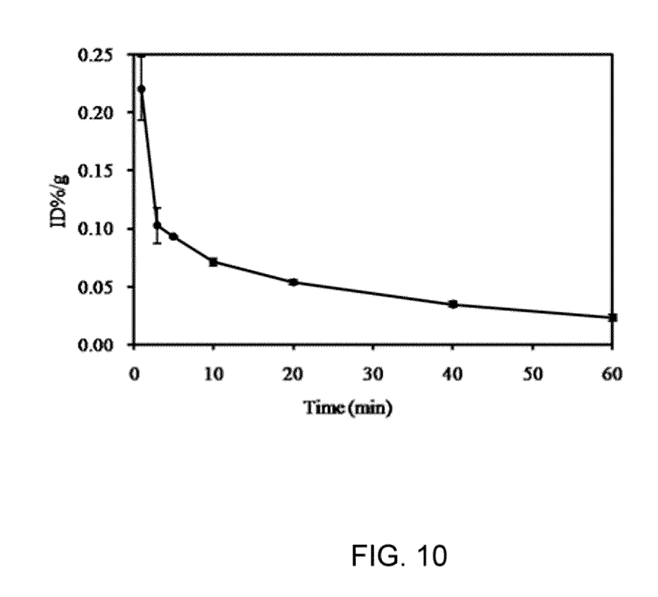

FIG. 10 illustrates blood clearance of ⁶⁴Cu-DOTA-C-ANF in rabbit (n=4). The activity in blood was 0.22% ID/g at 1 min p.i., and decreased to 0.02% ID/g at 60 min p.i. The results from gamma counting showed that at 1 min post injection (p.i.), the activity left in blood was 0.22% ID/g, which decreased sharply to 0.10% ID/g in 2 mins and declined slowly to 0.02% ID/g at 60 min p.i.

### Example 13

This example illustrates plasma cholesterol levels in the rabbit atherosclerosis model.

At the time of imaging, compared to the reported normal baseline value of total plasma cholesterol (72±12 mg/dL), the rabbits on a high-cholesterol diet had 1111±366 mg/dL, 1451±421 mg/dL, and 1554±265 mg/dL total plasma cholesterol at TP1, TP2 and TP3, respectively.

### Example 14

This example illustrates histopathology in the rabbit atherosclerosis model.

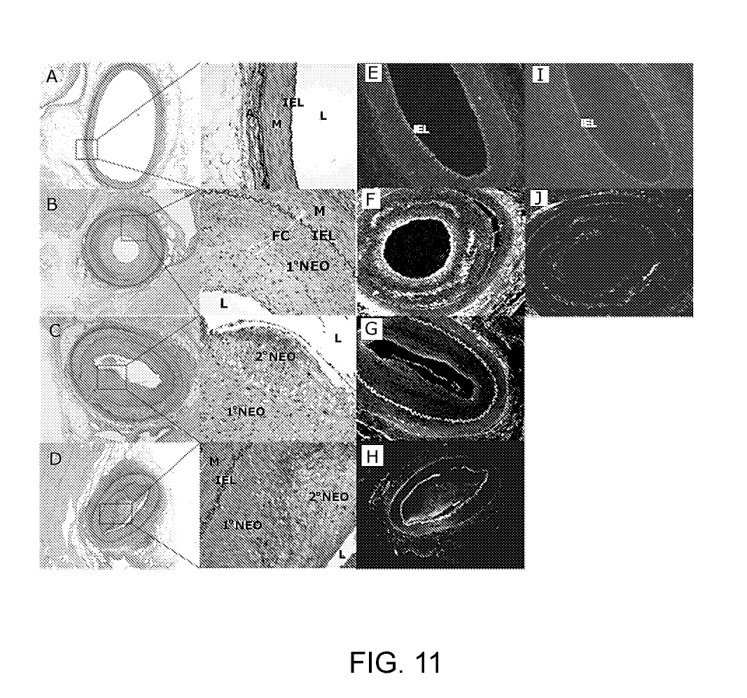

Light micrographs of femoral arterial cross-sections from hypercholesterolemic rabbits were obtained at time points after injury and stained with Verhoeff's Van Gieson (VVG) for elastin (FIG. 11A-D) as well as fluorescent images of corresponding sections immunostained for NPR-C (FIG. 11E-H) or blocked before immunostaining (FIGS. 11I and J). Low power micrographs are at 4×, high power insets are 400×. L=lumen. FIG. 11A. Uninjured, control femoral artery from the TP 2 rabbit shown in FIG. 11C. Inset shows intact internal elastic lamina (IEL), media (M), and adventitia (FIG. 11A). FIG. 11B: TP1 4 weeks after air desiccation-induced injury of a femoral artery. Inset shows a primary neointima (1° NEO) containing numerous foam cells (FC) and the dark nuclei of smooth muscle cells. FIG. 11C: TP2 4 weeks after balloon overstretch injury of a previous air-dessication-induced lesion showing a broken IEL (adjacent to inset box) and development of an amorphous, less cellular secondary neointima (2° NEO) shown in the inset. The endothelium is artifactually lifted away from the neointima. FIG. 11D: TP3 8 weeks after balloon overstretch injury showing enlarged 2° neointima comprised predominantly of matrix and elastin elements with few cells. FIG. 11E: Uninjured, control femoral artery showing only IEL autofluorescence. FIG. 11F: TP1 Showing increased fluorescence near the luminal or endothelial surface of the primary neointima (arrow). FIG. 11G, H: TP2 and TP3 Some fluorescence on the less cellular secondary neointima, but not as much as seen at TP1. In all immunostained images, the IEL demonstrates autoflorescence. FIG. FIG. 11I: Uninjured, control femoral artery immunofluorescence after competitive blocking of the antibody-antigen binding shows no difference in comparison to the pre-blocked image. FIG. 11J: TP1 immunofluorescence after competitive blocking of the antibody-antigen binding indicating the specific binding to NPR-C.

In these experiments, as shown in FIG. 11, histology of femoral artery sections obtained at TP1 confirmed the formation of primary neointimal lesions comprised of vascular smooth muscle cells, foam cells (FC), and extracellular matrix at the site of injury (FIG. 11B). The complexity of lesions that had undergone secondary angioplasty (femoral artery specimens obtained at TP 2 and TP 3) was increased in comparison to specimens that underwent air desiccation alone. Specimens obtained at TP 2 and 3 showed disruption of the internal elastic lamina (IEL) and a luminal stenotic lesion that was largely acellular (FIGS. 11C and D). These lesions had the appearance of restenotic lesions in human subjects who have undergone angioplasty. No evidence of fibrous cap thinning, fissures or thrombosis was observed to suggest instability. The sections obtained from the contralateral uninjured arteries showed no neointimal formation despite the presence of high cholesterol plasma (FIG. 11A).

Immunohistochemistry confirmed the presence of NPR-C on the luminal surface of the neointimal (FIG. 11). Staining was relatively increased in the previously injured arteries compared with the non-injured control arteries. NPR-C presence also appeared to be the highest in specimens obtained at TP1 (FIG. 11F) compared with TP 2 (FIG. 11G) and TP 3 (FIG. 11H). Separate competitive blocking studies demonstrated decreased immuno staining for NPR-C, further confirming the presence of the receptor.

### Example 15

This example illustrates MRI of Gadofluorine uptake in atherosclerotic-like lesions

In these experiments, the injured vessels in all rabbits at all time points demonstrated increased signal secondary to Gadofluorine M uptake. The site of injury visible on MRI also correlated with regions of increased radiotracer activity within the injured artery seen on PET. The control arteries showed no Gadofluorine M uptake.

### Example 16

This example illustrates quantitative PET imaging of ⁶⁴Cu-DOTA-C-ANF in atherosclerotic-like lesions.

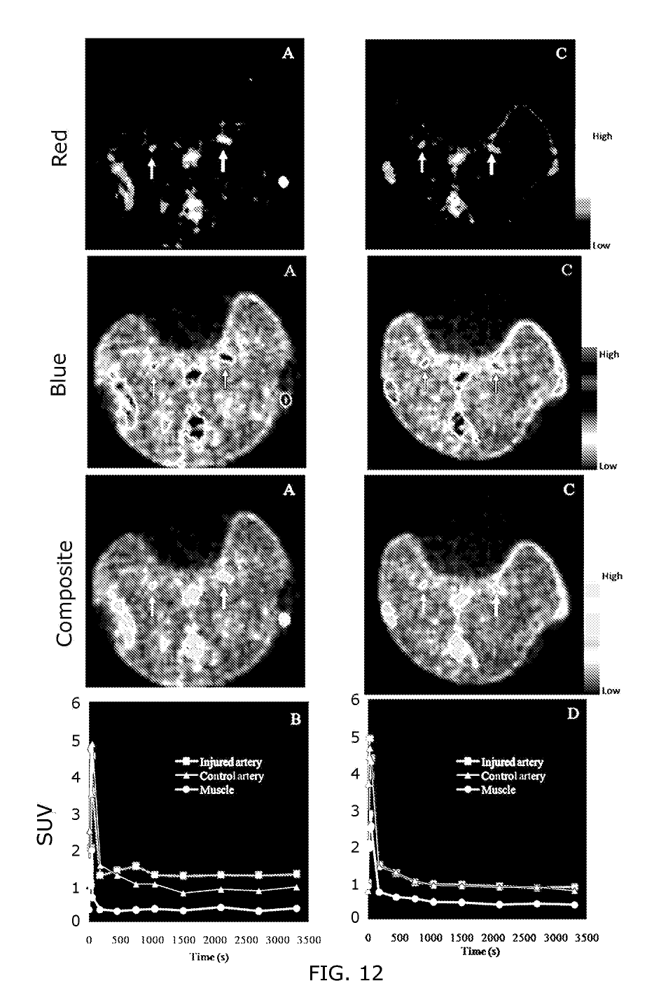

Specific binding of ⁶⁴Cu-DOTA-C-ANF on injured arteries from rabbits from time points (TPs) is demonstrated in FIG. 12. PET images (FIG. 12A, C) show ⁶⁴Cu-DOTA-C-ANF tracer uptake at the site of vessel injury and induced atherosclerosis with significant difference from non-injured control vessels in all animals. The original red channel, original blue channel, and a composite are shown.

In these experiments, tracer uptake in the injured femoral artery was visualized by PET. FIG. 12A is a PET image of a pre-blocking study. It shows a representative PET transverse slice from a rabbit injected with ⁶⁴Cu-DOTA-C-ANF (5-60 min summed frames). FIG. 12B shows a time activity curve of pre-blocking PET image of FIG. 12A. This time activity curve shows a stable uptake over the 1 h dynamic scan. FIG. 12C is a PET image of a blocking study. FIG. 12D is a time activity curve of PET blocking image FIG. 12C.

The muscle tracer uptake around the injured artery shows low background (FIG. 12A). On a representative transverse plane of the MAP reconstructed microPET images (FIG. 12A), the uptake of ⁶⁴Cu-DOTA-C-ANF tracer at the injured right femoral artery is evident (right arrow). The left femoral arteries were used as control and showed weak uptake (FIG. 12A, left arrow). On the representative transverse plane of microPET image with C-ANF peptide blocking (FIG. 12C), both injured artery (right arrow) and control artery (left arrow) showed similar weak uptake. The time activity curves (FIG. 12B, D) showed stable uptake in the injured arteries.

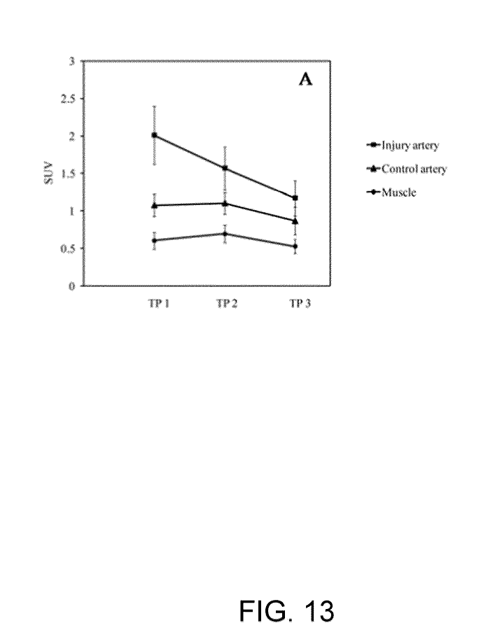

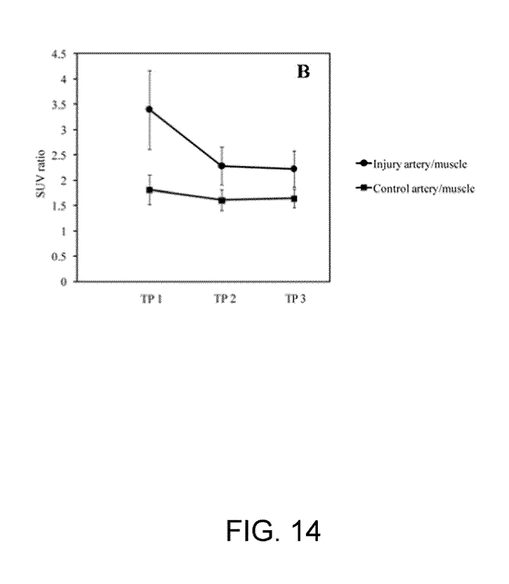

FIG. 13 illustrates ⁶⁴Cu-DOTA-C-ANF tracer uptake SUV on injured femoral arteries, non-injured control arteries, and surrounding muscle with the progression and remodeling of atherosclerotic plaques, and FIG. 14 illustrates target-to-background ratios of tracer uptake at the three time points studies (TP 1 (n=8), TP2 (n=6), TP3 (n=4)). The highest uptake on the luminal surface at the injury site was observed at TP 1 (SUV=2.01±0.39) and decreased at later time points (FIG. 13). Noticeably, the uptake of ⁶⁴Cu-DOTA-C-ANF on the injured vessel was significantly higher than that on the non-injured control artery (P<0.05) and muscle (P<0.005) at each considered time point. The target-to-background (injured artery/muscle) ratio reached the highest at TP 1 (3.39±0.78) (FIG. 14). This value decreased significantly at TP 2 and remained constant at TP 3 (P<0.05). On the contrary, no significant differences were observed in the control artery/muscle ratio at the three time points. Importantly, the injured artery/muscle SUV ratio was significantly higher than that of control artery/muscle at each time point (P<0.05).

Receptor blocking experiments (n=3) showed the similar uptakes on PET images for both injured artery and non-injured control artery (FIG. 12C), which was also illustrated in the time activity curve (FIG. 12D). The SUV ratio in the blocking studies of injured/control arteries was 1.07±0.06, significantly (P<0.05) lower than that (1.42±0.03) obtained in the pre-blocking studies. The target-to-background ratio on injured artery/muscle was reduced from 3.05±0.19 to 2.13±0.18 due to the competitive receptor blocking (P<0.01). In contrast, the control artery/muscle ratio was hardly altered (2.19±0.16 vs. 2.00±0.06). As a result of receptor blocking, the significant difference (P<0.001) between the injured artery/muscle and control artery/muscle in the pre-blocking studies was changed to no difference in the blocking studies. The average SUVs of injured arteries were declined from 1.69±0.31 to 1.18±0.18 (P<0.05) while the alteration of the control arteries was from 1.21±0.20 to 1.11±0.17 following the blocking studies. The uptake of ⁶⁴Cu-DOTA-C-ANF in muscle had an SUV of 0.56±0.11 and 0.55±0.09 before and after the blocking.

### Example 17

This example presents results obtained using 25% CANF-Comb PET in a rabbit model.

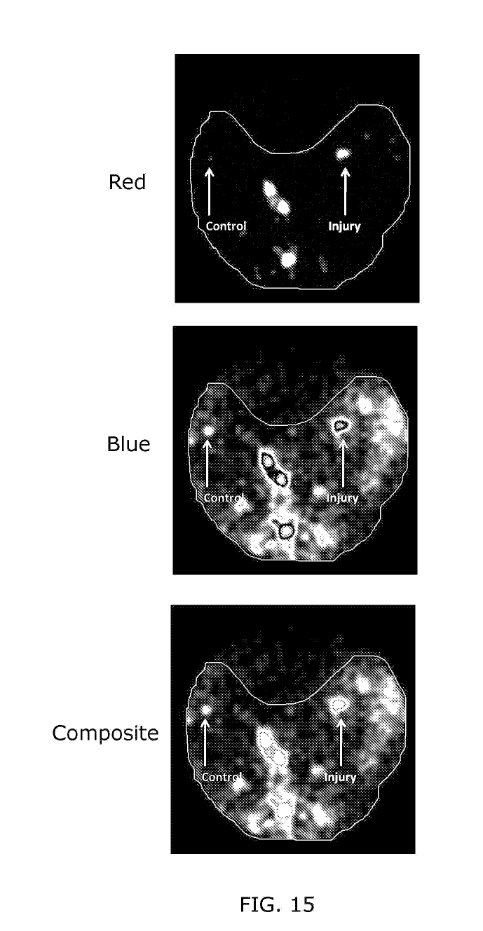

In these experiments, rabbits were injected with ⁶⁴Cu-CANF-Comb(˜2 mCi) at each time point. Animals were subjected to MicroPET scan at 1 h, 4 h, and 24 h post injection on Focus 220 scanner. At each time point, IHC and histopathology were performed on injured and control arteries to assess NPR-C presence and plaque morphology. FIG. 15 shows a representative PET scan using ⁶⁴Cu labeling Of 25% CANF-Comb. Specific activity was 70 μCi/γg; Dose 1.8 mCi. The results of the experiments are summarized as following table.

| | | | | ⁶⁴Cu-DOTA-CANF |
|---|---|---|---|---|
| | ⁶⁴Cu-CANF-Comb (n = 4) | | | (n = 9) |
| Time | 1 h | 4 h | 24 h | 1 h |
| Injury SUV | 5.31 ± 1.51 | 4.76 ± 1.92 | 3.74 ± 2.01 | 2.01 ± 0.39 |
| Control SUV | 1.30 ± 0.38 | 1.15 ± 0.32 | 0.88 ± 0.17 | 0.94 ± 0.27 |
| Injury/control | 4.07 ± 0.20 | 4.07 ± 0.33 | 4.24 ± 0.29 | 1.87 ± 0.29 |
| SUV ratios | | | | |
| Injury/muscle | 38.2 ± 11.2 | 37.7 ± 12.4 | 40.8 ± 10.6 | 3.39 ± 0.78 |
| SUV ratios | | | | |

The data demonstrate higher injury/control SUV values and higher injury/muscle SUV values for ⁶⁴Cu-CANF-Comb compared to ⁶⁴Cu-DOTA-CANF.

All references cited herein are incorporated by reference, each in its entirety.

What is claimed is:

1. A tracer comprising:
an amphiphilic comb-like nanostructure conjugated with an oligopeptide comprising a fragment of a natriuretic peptide, wherein the fragment comprises Arg-Ile-Asp-Arg-Ile (SEQ ID NO:1); and
a positron-emitting radionuclide.

2. A tracer in accordance with claim 1, wherein the fragment of a natriuretic peptide comprises Cys-Phe-Gly-Gly-Arg-Ile-Asp-Arg-Ile-Gly-Ala-Cys-NH₂, (SEQ ID NO:2).

3. A tracer in accordance with claim 1, wherein the fragment of a natriuretic peptide comprises H-Arg-Ser-Ser-Cys-Phe-Gly-Gly-Arg-Ile-Asp-Arg-Ile-Gly-Ala-Cys-NH₂ (SEQ ID NO:3).

4. A tracer in accordance with claim 1, wherein the positron-emitting radionuclide selected from the group consisting of carbon-11, nitrogen-13, oxygen-14, oxygen-15, fluorine-18, iron-52, copper-62, copper-64, zinc-62 zinc-63, gallium-68, arsenic-74, bromine-76, rubidium-82, yttrium-86, zirconium-89, technetium-94m, indium-110m, iodine-122, iodine-123, iodine-124, iodine-131 and cesium-137.

5. A tracer in accordance with claim 4, wherein the positron-emitting radionuclide is selected from the group consisting of carbon-11, nitrogen-13, oxygen-15, fluorine-18, iron-52, copper-64, gallium-68, yttrium-86, bromine-76, zirconium-89, iodine-123, and iodine-124.

6. A tracer in accordance with claim 5, wherein the positron-emitting radionuclide is selected from the group consisting of carbon-11, nitrogen-13, oxygen-15, fluorine-18, and copper-64.

7. A tracer in accordance with claim 6, wherein the radionuclide is a copper-64.

8. A tracer in accordance with claim 7, further comprising a radionuclide carrier moiety.

9. A tracer in accordance with claim 8, wherein the carrier moiety is a 1,4,7,10-tetraazacyclododecane-1,4,7,10-tetraacetic acid (DOTA).

10. A tracer in accordance with claim 1, wherein the fragment of the natriuretic peptide consists of no more than 19 amino acids.

11. A method of determining distribution of C-type atrial natriuretic peptide receptors in a subject, comprising:
administering to the subject a tracer comprising a) an amphiphilic comb-like nanostructure conjugated with an oligopeptide comprising a fragment of a natriuretic peptide, wherein the fragment comprises Arg-Ile-Asp-Arg-Ile (SEQ ID NO:1) and b) a positron-emitting radionuclide; and
subjecting the subject to positron emission tomography scanning.

12. A method in accordance with claim 11, wherein the fragment of on natriuretic peptide comprises Cys-Phe-Gly-Gly-Arg-Ile-Asp-Arg-Ile-Gly-Ala-Cys-NH₂, (SEQ ID NO:2).

13. A method in accordance with claim 11, fragment of a natriuretic peptide comprises H-Arg-Ser-Ser-Cys-Phe-Gly-Gly-Arg-Ile-Asp-Arg-Ile-Gly-Ala-Cys-NH₂ (SEQ ID NO:3).

14. A tracer in accordance with claim 11, wherein the fragment of the natriuretic peptide consists of no more than 19 amino acids.

15. A method in accordance with claim 11, wherein the position-emitting radionuclide is selected from the group consisting of carbon-11, nitrogen-13; oxygen-14, oxygen-15, fluorine-18, iron-52, copper-62, copper-64, zinc-62 zinc-63, gallium-68, arsenic-74, bromine-76, rubidium-82, yttrium-86, zirconium-89, technetium-94m, indium-110m, iodine-122, iodine-123, iodine-124, iodine-131 and cesium-137.

16. A method in accordance with claim 16, wherein the positron-emitting radionuclide is a copper-64.

17. A method of imaging angiogenesis or atherosclerosis in a subject, comprising:
administering to the subject a tracer comprising a) an amphiphilic comb-like nanostructure conjugated with an oligopeptide comprising a fragment of a natriuretic peptide, wherein the fragment comprises Arg-Ile-Asp-Arg-Ile (SEQ ID NO:1) and b) a positron-emitting radionuclide; and
subjecting the subject to positron emission tomography scanning.

18. A method in accordance with claim 17, wherein the fragment of a natriuretic peptide comprises Cys-Phe-Gly-Gly-Arg-Ile-Asp-Arg-Ile-Gly-Ala-Cys-NH₂, (SEQ ID NO:2).

19. A method in accordance with claim 18, fragment of a natriuretic peptide comprises H-Arg-Ser-Ser-Cys-Phe-Gly-Gly-Arg-Ile-Asp-Arg-Ile-Gly-Ala-Cys-NH₂ (SEQ ID NO:3).

20. A method in accordance with claim 17, wherein the positron-emitting radionuclide is copper-64.

21. A tracer in accordance with claim 17, wherein the fragment of the natriuretic peptide consists of no more than 19 amino acids.
